(19) United States
(12) Patent Application Publication
(10) Pub. No.: US 2026/0345103 A1
(43) Pub. Date: Nov. 5, 2026

(54) GENERATION OF SYNTHETIC RADIOLOGICAL IMAGES

(71) Applicant: BAYER AKTIENGESELLSCHAFT, Leverkusen (DE)

(72) Inventors: Matthias LENGA, Leverkusen (DE); Ivo Matteo BALTRUSCHAT, Leverkusen (DE); Felix Karl KREIS, Leverkusen (DE)

(21) Appl. No.: 19/107,223

(22) PCT Filed: Aug. 23, 2023
(86) PCT No.: PCT/EP2023/073102

§ 371 Date: Feb. 27, 2025

(30) Foreign Application Priority Data
Aug. 30, 2022 (EP) 22192907.8
Mar. 1, 2023 (EP) 23159288.2

(51) Int. Cl. A61B 6/00; A61K 49/10; G06T 7/00

(52) U.S. Cl. CPC A61B 6/481; A61B 6/5205; A61K 49/108; G06T 7/0012; G06T 2207/10116; G06T 2207/20081; G06T 2207/20084

(57) ABSTRACT

The present invention relates to the technical field of radiology, and in particular to assisting radiologists in radiological examinations using artificial intelligence methods. The present invention relates to training a machine learning model and using the trained model to predict representations of an examination area after applications of different amounts of contrast agent.

### TECHNICAL FIELD

The present invention relates to the technical field of radiology, in particular that of assisting radiologists in radiological examinations using artificial intelligence methods. The present invention is concerned with the training of a machine-learning model and the utilization of the trained model for predicting representations of an examination region after administration of different amounts of a contrast agent.

### INTRODUCTION

WO2019/074938A1 discloses a method for reducing the amount of contrast agent in the generation of radiological images with the aid of an artificial neural network.

In a first step, a training data set is generated. The training data set comprises for each person of a multiplicity of persons i) a native radiological image (zero-contrast image), ii) a radiological image after administration of a low amount of contrast agent (low-contrast image) and iii) a radiological image after administration of a standard amount of contrast agent (full-contrast image).

In a second step, an artificial neural network is trained to predict for each person of the training data set, on the basis of the native image and the image after administration of a low amount of contrast agent, an artificial radiological image showing an acquisition region after administration of the standard amount of contrast agent. The measured radiological image after administration of a standard amount of contrast agent serves in each case as reference (ground truth) in the training.

In a third step, the trained artificial neural network can be used to generate for a new person, on the basis of a native image and of a radiological image after administration of a low amount of contrast agent, a synthetic radiological image showing the acquired region as it would look if a standard amount of contrast agent had been administered.

It has been found that the synthetic radiological images generated in this way may contain artifacts. Either fine structures may not be correctly reproduced, or regions of the synthetic radiological image may contain structures not found in the represented tissue (see for example: K. Schwarz et al.: On the Frequency Bias of Generative Models, https://doi.org/10.48550/arXiv.2111.02447).

### SUMMARY

This and other problems are addressed by the present disclosure.

The present disclosure provides in a first aspect a computer-implemented method for training a machine-learning model (training method). The training method comprises:
receiving and/or providing training data, the training data comprising a set of input data and target data for each examination object of a multiplicity of examination objects,
   each set comprising at least one input representation of an examination region of the examination object as input data and a target representation of the examination region of the examination object and a transformed target representation as target data,
   the at least one input representation representing the examination region without contrast agent and/or after administration of a first amount of a contrast agent and the target representation representing the examination region after the administration of a second amount of the contrast agent,
   the transformed target representation representing at least part of the examination region of the examination object
      in frequency space, if the target representation represents the examination region of the examination object in real space, or
      in real space, if the target representation represents the examination region of the examination object in frequency space,
receiving and/or providing a machine-learning model, the machine-learning model being configured to generate on the basis of at least one input representation of an examination region of an examination object and model parameters a synthetic representation of the examination region of the examination object,
training the machine-learning model, the training comprising for each examination object of the multiplicity of examination objects:
   feeding the at least one input representation of the examination region of the examination object to the machine-learning model,
   receiving a synthetic representation of the examination region of the examination object from the machine-learning model,
   generating and/or receiving a transformed synthetic representation on the basis of the synthetic representation and/or in relation to the synthetic representation, the transformed synthetic representation representing at least part of the examination region of the examination object
      in frequency space, if the synthetic representation represents the examination region of the examination object in real space, or
      in real space, if the synthetic representation represents the examination region of the examination object in frequency space,
   quantifying the differences i) between at least part of the synthetic representation and at least part of the target representation and ii) between at least part of the transformed synthetic representation and at least part of the transformed target representation by means of a loss function,
   reducing the differences by modifying model parameters,
outputting and/or storing the trained machine-learning model and/or the model parameters and/or transmitting the trained machine-learning model and/or the model parameters to a separate computer system and/or using the trained machine-learning model for generation of a synthetic representation of the examination region of a new examination object.

The present disclosure further provides a computer-implemented method for generating a synthetic radiological image (prediction method). The prediction method comprises:
providing a trained machine-learning model,
   the trained machine-learning model having been trained by means of training data to generate on the basis of at least one input representation of an examination region of an examination object a synthetic representation of the examination region of the examination object, the at least one input representation representing the examination region without contrast agent and/or after administration of a first amount of a contrast agent and the synthetic representation representing the examination region after the administration of a second amount of the contrast agent,
   the training data comprising for each examination object of a multiplicity of examination objects i) at least one input representation of the examination region of the examination object, ii) a target representation of the examination region and iii) a transformed target representation,
   the transformed target representation representing at least part of the examination region in frequency space, if the target representation represents the examination region in real space, or in real space, if the target representation represents the examination region in frequency space,
   the training of the machine-learning model comprising reducing differences between i) at least part of the synthetic representation and at least part of the target representation and ii) between at least part of a transformed synthetic representation and at least part of the transformed target representation,
receiving at least one input representation of an examination region of a new examination object, the at least one input representation representing the examination region of the new examination object without contrast agent and/or after administration of a first amount of a contrast agent,
inputting the at least one input representation of the examination region of the new examination object into the trained machine-learning model,
receiving a synthetic representation of the examination region of the new examination object from the machine-learning model,
outputting and/or storing the synthetic representation of the examination region of the new examination object and/or transmitting the synthetic representation of the examination region of the new examination object to a separate computer system.

The present disclosure further provides a computer system comprising
a receiving unit,
a control and calculation unit and
an output unit,
   wherein the control and calculation unit is configured to provide a trained machine-learning model,
      the trained machine-learning model having been trained by means of training data to generate on the basis of at least one input representation of an examination region of an examination object a synthetic representation of the examination region,
      the training data comprising for each examination object of a multiplicity of examination objects i) an input representation of the examination region of the examination object, ii) a target representation of the examination region and iii) a transformed target representation,
         the at least one input representation representing the examination region of the respective examination object without contrast agent or after administration of a first amount of a contrast agent,
         the target representation representing the examination region of the respective examination object after administration of a second amount of the contrast agent,
         the transformed target representation representing at least part of the examination region of the respective examination object in frequency space, if the target representation represents the examination region of the respective examination object in real space, or in real space, if the target representation represents the examination region of the respective examination object in frequency space,
      the training of the machine-learning model comprising reducing differences between i) at least part of the synthetic representation and at least part of the target representation and ii) at least part of a transformed synthetic representation and at least part of the transformed target representation,
   wherein the control and calculation unit is configured to cause the receiving unit to receive at least one input representation of an examination region of a new examination object, the at least one input representation representing the examination region without and/or after administration of a first amount of a contrast agent,
   wherein the control and calculation unit is configured to input the received at least one input representation into the trained machine-learning model,
   wherein the control and calculation unit is configured to receive from the trained machine-learning model a synthetic representation of the examination region of the new examination object,
   wherein the control and calculation unit is configured to cause the output unit to output the synthetic representation of the examination region of the new examination object and/or to store it and/or to transmit it to a separate computer system.

The present disclosure further provides a computer program product comprising a computer program which can be loaded into a working memory of a computer system, where it causes the computer system to execute the following steps:
providing a trained machine-learning model,
   the trained machine-learning model having been trained by means of training data to generate on the basis of at least one input representation of an examination region of an examination object a synthetic representation of the examination region of the examination object,
   the training data comprising for each examination object of a multiplicity of examination objects i) an input representation of the examination region of the examination object, ii) a target representation of the examination region of the examination object and iii) a transformed target representation,
      the at least one input representation representing the examination region of the respective examination object without contrast agent or after administration of a first amount of a contrast agent,
      the target representation representing the examination region of the respective examination object after administration of a second amount of the contrast agent,
      the transformed target representation representing at least part of the examination region of the examination object in frequency space, if the target representation represents the examination region of the examination object in real space, or in real space, if the target representation represents the examination region of the examination object in frequency space,
   the training of the machine-learning model comprising reducing the differences between i) at least part of the synthetic representation and at least part of the target representation and ii) between at least part of a transformed synthetic representation and at least part of the transformed target representation,
receiving at least one input representation of an examination region of anew examination object, the at least one input representation representing the examination region of the new examination object without contrast agent and/or after administration of a first amount of a contrast agent,
inputting the at least one input representation of the examination region of the new examination object into the trained machine-learning model,
receiving a synthetic representation of the examination region of the new examination object from the trained machine-learning model,
outputting and/or storing the synthetic representation of the examination region of the new examination object and/or transmitting the synthetic representation of the examination region of the new examination object to a separate computer system.

The present disclosure further provides for the use of a contrast agent in a radiological examination method, the radiological examination method comprising:
providing a trained machine-learning model,
   the trained machine-learning model having been trained by means of training data to generate on the basis of at least one input representation of an examination region of an examination object a synthetic representation of the examination region of the examination object,
   the training data comprising for each examination object of a multiplicity of examination objects i) an input representation of the examination region of the examination object, ii) a target representation of the examination region of the examination object and iii) a transformed target representation,
      the at least one input representation representing the examination region of the respective examination object without or after administration of a first amount of the contrast agent,
      the target representation representing the examination region of the respective examination object after administration of a second amount of the contrast agent,
      the transformed target representation representing at least part of the examination region of the examination object in frequency space, if the target representation represents the examination region of the examination object in real space, or in real space, if the target representation represents the examination region of the examination object in frequency space,
   the training of the machine-learning model comprising reducing the differences between i) at least part of the synthetic representation and at least part of the target representation and ii) between at least part of a transformed synthetic representation and at least part of the transformed target representation,
receiving at least one input representation of an examination region of anew examination object, the at least one input representation representing the examination region of the new examination object without contrast agent and/or after administration of a first amount of a contrast agent,
inputting the at least one input representation of the examination region of the new examination object into the trained machine-learning model,
receiving a synthetic representation of the examination region of the new examination object from the trained machine-learning model,
outputting and/or storing the synthetic representation of the examination region of the new examination object and/or transmitting the synthetic representation of the examination region of the new examination object to a separate computer system.

The present disclosure further provides a contrast agent for use in a radiological examination method, the radiological examination method comprising:
providing a trained machine-learning model,
   the trained machine-learning model having been trained by means of training data to generate on the basis of at least one input representation of an examination region of an examination object a synthetic representation of the examination region of the examination object,
   the training data comprising for each examination object of a multiplicity of examination objects i) an input representation of the examination region of the examination object, ii) a target representation of the examination region of the examination object and iii) a transformed target representation,
      the at least one input representation representing the examination region of the respective examination object without or after administration of a first amount of the contrast agent,
      the target representation representing the examination region of the respective examination object after administration of a second amount of the contrast agent,
      the transformed target representation representing at least part of the examination region of the examination object in frequency space, if the target representation represents the examination region of the examination object in real space, or in real space, if the target representation represents the examination region of the examination object in frequency space,
   the training of the machine-learning model comprising reducing the differences between i) at least part of the synthetic representation and at least part of the target representation and ii) between at least part of a transformed synthetic representation and at least part of the transformed target representation,
receiving at least one input representation of an examination region of anew examination object, the at least one input representation representing the examination region of the new examination object without contrast agent and/or after administration of a first amount of a contrast agent,
inputting the at least one input representation of the examination region of the new examination object into the trained machine-learning model,
receiving a synthetic representation of the examination region of the new examination object from the trained machine-learning model,
outputting and/or storing the synthetic representation of the examination region of the new examination object and/or transmitting the synthetic representation of the examination region of the new examination object to a separate computer system.

The present disclosure further provides a kit comprising a contrast agent and a computer program which can be loaded into a working memory of a computer system, where it causes the computer system to execute the following steps:
providing a trained machine-learning model,
   the trained machine-learning model having been trained by means of training data to generate on the basis of at least one input representation of an examination region of an examination object a synthetic representation of the examination region of the examination object,
   the training data comprising for each examination object of a multiplicity of examination objects i) an input representation of the examination region of the examination object, ii) a target representation of the examination region of the examination object and iii) a transformed target representation,
      the at least one input representation representing the examination region of the respective examination object without or after administration of a first amount of the contrast agent,
      the target representation representing the examination region of the respective examination object after administration of a second amount of the contrast agent,
      the transformed target representation representing at least part of the examination region of the examination object in frequency space, if the target representation represents the examination region of the examination object in real space, or in real space, if the target representation represents the examination region of the examination object in frequency space,
   the training of the machine-learning model comprising reducing the differences between i) at least part of the synthetic representation and at least part of the target representation and ii) between at least part of a transformed synthetic representation and at least part of the transformed target representation,
receiving at least one input representation of an examination region of anew examination object, the at least one input representation representing the examination region of the new examination object without contrast agent and/or after administration of a first amount of a contrast agent,
inputting the at least one input representation of the examination region of the new examination object into the trained machine-learning model,
receiving a synthetic representation of the examination region of the new examination object from the trained machine-learning model,
outputting and/or storing the synthetic representation of the examination region of the new examination object and/or transmitting the synthetic representation of the examination region of the new examination object to a separate computer system.

Further subjects and embodiments can be found in the following description, the claims and the drawings.

### BRIEF DESCRIPTION OF THE DRAWINGS

FIG. 1 shows by way of example and in schematic form the process of training the machine-learning model.

FIG. 2 shows by way of example and in schematic form a use of a trained machine-learning model for prediction.

FIG. 3 shows by way of example and in schematic form a further embodiment of the training of the machine-learning model.

FIG. 4 shows by way of example and in schematic form a further embodiment of the training of the machine-learning model.

FIG. 5 shows by way of example and in schematic form a further embodiment of the training of the machine-learning model.

FIG. 6 shows by way of example and in schematic form a further embodiment of the training of the machine-learning model.

FIG. 7 shows by way of example and in schematic form a further embodiment of the training of the machine-learning model.

FIG. 8 shows by way of example and in schematic form a further embodiment of the training of the machine-learning model.

FIG. 9 shows by way of example and in schematic form a further embodiment of the training of the machine-learning model.

FIG. 10 shows schematically an example of the use of the trained machine-learning model for prediction.

FIG. 11 shows the result of validation (a) of the machine-learning model trained according to FIG. 8 and (b) of the machine-learning model trained according to FIG. 9.

FIG. 12 shows by way of example and in schematic form a computer system according to the present disclosure.

FIG. 13 shows by way of example and in schematic form a further embodiment of the computer system according to the present disclosure.

FIG. 14 shows schematically in the form of a flow chart one embodiment of the method for training a machine-learning model.

FIG. 15 shows schematically in the form of a flow chart one embodiment of the method for generating a synthetic representation of an examination region of an examination object with the aid of the trained machine-learning model.

### DETAILED DESCRIPTION

The invention will be more particularly elucidated below without distinguishing between the subjects of the invention (training method, prediction method, computer system, computer program, use, contrast agent for use, kit). Rather, the following remarks are intended to apply mutatis mutandis to all subjects (training method, prediction method, computer system, computer program, use, contrast agent for use, kit), irrespective of the context in which the remarks are made.

Where steps are stated in an order in the present description or in the claims, this does not necessarily mean that the invention is limited to the order stated. Instead, it is conceivable that the steps are also executed in a different order or else in parallel with one another, the exception being when one step builds on another step, thereby making it imperative that the step building on the previous step be executed next (which will however become clear in the individual case). The orders stated are thus preferred embodiments of the invention.

In certain places the invention will be more particularly elucidated with reference to drawings. The drawings show specific embodiments having specific features and combinations of features, which are intended primarily for illustrative purposes; the invention is not to be understood as being limited to the features and combinations of features shown in the drawings. Furthermore, statements made in the description of the drawings in relation to features and combinations of features are intended to be generally applicable, that is to say applicable to other embodiments too and not limited to the embodiments shown.

With the aid of the present invention, representations of an examination region of an examination object can be predicted. Such a predicted representation is also referred to as a synthetic representation in this disclosure.

The “examination object” is normally a living being, preferably a mammal, most preferably a human.

The “examination region” is part of the examination object, for example an organ or part of an organ, such as the liver, the brain, the heart, the kidney, the lung, the stomach, the intestines, the pancreas, the thyroid gland, the prostate, the breast or part of the aforementioned organs, or multiple organs or another part of the body.

In one embodiment, the examination region includes a liver or part of a liver or the examination region is a liver or part of a liver of a mammal, preferably a human.

In a further embodiment, the examination region includes a brain or part of a brain or the examination region is a brain or part of a brain of a mammal, preferably a human.

In a further embodiment, the examination region includes a heart or part of a heart or the examination region is a heart or part of a heart of a mammal, preferably a human.

In a further embodiment, the examination region includes a thorax or part of a thorax or the examination region is a thorax or part of a thorax of a mammal, preferably a human.

In a further embodiment, the examination region includes a stomach or part of a stomach or the examination region is a stomach or part of a stomach of a mammal, preferably a human.

In a further embodiment, the examination region includes a pancreas or part of a pancreas or the examination region is a pancreas or part of a pancreas of a mammal, preferably a human.

In a further embodiment, the examination region includes a kidney or part of a kidney or the examination region is a kidney or part of a kidney of a mammal, preferably a human.

In a further embodiment, the examination region includes one or both lungs or part of a lung of a mammal, preferably a human.

In a further embodiment, the examination region includes a breast or part of a breast or the examination region is a breast or part of a breast of a female mammal, preferably a female human.

In a further embodiment, the examination region includes a prostate or part of a prostate or the examination region is a prostate or part of a prostate of a male mammal, preferably a male human.

The examination region, also referred to as the field of view (FOV), is in particular a volume that is imaged in radiological images. The examination region is typically defined by a radiologist, for example on a localizer image. It is of course also possible for the examination region to be alternatively or additionally defined in an automated manner, for example on the basis of a selected protocol.

A “representation of the examination region” is normally the result of a radiological examination.

“Radiology” is the branch of medicine that is concerned with the use of predominantly electromagnetic rays and mechanical waves (including for instance ultrasound diagnostics) for diagnostic, therapeutic and/or scientific purposes. Besides X-rays, other ionizing radiation such as gamma radiation or electrons are also used. Imaging being a key application, other imaging methods such as sonography and magnetic resonance imaging (nuclear magnetic resonance imaging) are also counted as radiology, even though no ionizing radiation is used in these methods. The term “radiology” in the context of the present invention thus encompasses in particular the following examination methods: computed tomography, magnetic resonance imaging, sonography.

In one embodiment of the present disclosure, the radiological examination is a magnetic resonance imaging examination.

In a further embodiment, the radiological examination is a computed tomography examination.

In one embodiment, the radiological examination is an ultrasound examination.

In radiological examinations, contrast agents are commonly used for contrast enhancement.

“Contrast agents” are substances or mixtures of substances that improve the depiction of structures and functions of the body in radiological examinations.

In computed tomography, iodine-containing solutions are normally used as contrast agents. In magnetic resonance imaging (MRI), superparamagnetic substances (e.g. iron oxide nanoparticles, superparamagnetic iron-platinum particles (SIPPs)) or paramagnetic substances (e.g. gadolinium chelates, manganese chelates) are usually used as contrast agents. In the case of sonography, liquids containing gas-filled microbubbles are usually administered intravenously. Examples of contrast agents can be found in the literature (see for example A. S. L. Jascinth et al.: Contrast Agents in computed tomography: A Review, Journal of Applied Dental and Medical Sciences, 2016, vol. 2, issue 2, 143-149; H. Lusic et al.: X-ray-Computed Tomography Contrast Agents, Chem. Rev. 2013, 113, 3, 1641-1666; https://www.radiology.wisc.edu/wp-content/uploads/2017/10/contrast-agents-tutorial.pdf, M. R. Nouh et al.: Radiographic and magnetic resonances contrast agents: Essentials and tips for safe practices, World J Radiol. 2017 Sep. 28; 9(9): 339-349; L. C. Abonyi et al.: Intravascular Contrast Media in Radiography: Historical Development & Review of Risk Factors for Adverse Reactions, South American Journal of Clinical Research, 2016, vol. 3, issue 1, 1-10; ACR Manual on Contrast Media, 2020, ISBN: 978-1-55903-012-0; A. Ignee et al.: Ultrasound contrast agents, Endosc Ultrasound. 2016 Nov-Dec; 5(6): 355-362).

MRI contrast agents exert their effect in an MRI examination by altering the relaxation times of the structures that take up contrast agents. A distinction can be made between two groups of substances: paramagnetic and superparamagnetic substances. Both groups of substances have unpaired electrons that induce a magnetic field around the individual atoms or molecules. Superparamagnetic contrast agents result in a predominant shortening of T2, whereas paramagnetic contrast agents mainly result in a shortening of T1. The effect of said contrast agents is indirect, since the contrast agent does not itself emit a signal, but instead merely influences the intensity of signals in its vicinity. An example of a superparamagnetic contrast agent is iron oxide nanoparticles (SPIO, superparamagnetic iron oxide). Examples of paramagnetic contrast agents are gadolinium chelates such as gadopentetate dimeglumine (trade name: Magnevist® and others), gadoteric acid (Dotarem®, Dotagita®, Cyclolux®), gadodiamide (Omniscan®), gadoteridol (ProHance®), gadobutrol (Gadovist®), and gadoxetic acid (Primovist®/Eovist®).

A representation of the examination region in the context of the present disclosure may be an MRI image, a CT image, an ultrasound image or the like, or the representation of the examination region may be generated from one or more MRI images, CT images, ultrasound images or the like.

A representation of the examination region in the context of the present disclosure may be a representation in real space (image space) or a representation in frequency space.

In a representation in real space, also referred to in this description as real-space depiction or real-space representation, the examination region is normally represented by a large number of image elements (pixels or voxels) that may for example be in a raster arrangement, in which case each image element represents a part of the examination region. A format widely used in radiology for storing and processing representations in real space is the DICOM format. DICOM (Digital Imaging and Communications in Medicine) is an open standard for storing and exchanging information in medical image data management.

In a representation in frequency space, also referred to in this description as frequency-space depiction or frequency-space representation, the examination region is represented by a superposition of fundamental frequencies. For example, the examination region may be represented by a sum of sine and cosine functions having different amplitudes, frequencies and phases. The amplitudes and phases may be plotted as a function of the frequencies, for example, in a two- or three-dimensional representation. Normally, the lowest frequency (origin) is placed in the center. The further away from this center, the higher the frequencies. Each frequency can be assigned an amplitude representing the frequency in the frequency-space depiction and a phase indicating the extent of the shift of the respective wave with respect to a sine or cosine wave.

A representation in real space can for example be converted (transformed) by a Fourier transform into a representation in frequency space. Conversely, a representation in frequency space can for example be converted (transformed) by an inverse Fourier transform into a representation in real space.

Details about real-space depictions and frequency-space depictions and their respective interconversion are described in numerous publications, see for example https://see.stanford.edu/materials/lsoftaee261/book-fall-07.pdf.

With the aid of a trained machine-learning model, it is possible to generate on the basis of at least one representation of an examination region of an examination object a synthetic representation of the examination region of the examination object. The at least one representation, on the basis of which the (trained) machine-learning model generates the synthetic representation, is also referred to in this description as an input representation.

The at least one input representation represents the examination region of an examination object before and/or after administration of a contrast agent. The synthetic representation represents the same examination region of the same examination object after administration of a second amount of the contrast agent. The first amount of the contrast agent and the second amount of the contrast agent are different from one another. In a preferred embodiment, the second amount is larger than the first amount.

The expression “after the second amount of a contrast agent” should not be understood as meaning that the first amount and the second amount add up in the examination region. Thus, the expression “the representation represents the examination region after administration of a (first or second) amount” should rather be understood as meaning: “the representation represents the examination region with a (first or second) amount” or “the representation represents the examination region including a (first or second) amount”.

In a preferred embodiment, the first amount of the contrast agent is smaller than the standard amount. The second amount of the contrast agent may correspond to the standard amount; alternatively, it may be smaller or larger. The standard amount is normally the amount recommended by the manufacturer and/or distributor of the contrast agent and/or the amount authorized by a regulatory authority and/or the amount specified in a package leaflet for the contrast agent. For example, the standard amount of Primovist® is thus 0.025 mmol Gd-EOB-DTPA disodium/kg body weight.

In the aforementioned embodiment, the machine-learning model of the present disclosure is thus configured and trained to generate, on the basis of at least one input representation of an examination region of an examination object representing the examination region before and/or after the administration of a first amount of contrast agent, a synthetic representation representing the examination region after the administration of a second amount of contrast agent, the first amount being preferably lower than the second amount. Such a trained machine-learning model may be used, for example, for reducing the amount of contrast agent in radiological examinations, as described in WO2019/074938A1. Such a trained machine-learning model may be used for converting a radiological image generated after the administration of a first (lower) amount of a contrast agent into a radiological image which, with regard to contrast distribution, has the appearance of a radiological image after the administration of a second (larger) amount of the contrast agent. In other words, the machine-learning model may be trained to produce contrast enhancement without the need to increase the amount of contrast agent.

The synthetic representation of the examination region representing the examination region after administration of a second amount of a contrast agent may be generated, for example, on the basis of a first input representation representing the examination region without contrast agent and a second input representation representing the examination region after the administration of a first amount of contrast agent. The first amount may be smaller than the second amount. The second amount may be the standard amount of the contrast agent. Alternatively, the second amount may be larger than the standard amount of the contrast agent.

For example, the first amount may be 90% or 80% or 70% or 60% or 50% or 40% or 30% or 20% or 10% of the second amount or less than 10% of the second amount or some other percentage of the second amount.

It is also possible that the at least one input representation comprises at least one first input representation and at least one second input representation. The at least one first input representation may represent the examination region after administration of a first amount of a contrast agent. The at least one second input representation may represent the examination region after administration of a second amount of a contrast agent. The first amount and the second amount may be different, for example the first amount may be smaller than the second amount. What may be generated on the basis of the at least one first input representation and the at least one second input representation is a synthetic representation representing the examination region after the administration of a third amount of the contrast agent. The third amount may be different from the first amount and the second amount, for example the third amount may be larger than the first and the second amount.

It is also possible that the at least one input representation comprises at least one first input representation, at least one second input representation and at least one third input representation. The at least one first input representation may represent the examination region after administration of a first amount of a contrast agent, it being possible for the first amount to also be zero. The at least one second input representation may represent the examination region after administration of a second amount of a contrast agent. The at least one third input representation may represent the examination region after administration of a third amount of a contrast agent. The first amount, the second amount and the third amount may be different, for example the first amount may be smaller than the second amount and the second amount may be smaller than the third amount. What may be generated on the basis of the at least one first input representation, the at least one second input representation and the at least one third input representation is a synthetic representation representing the examination region after the administration of a fourth amount of the contrast agent. The fourth amount may be different from the first amount, the second amount and the third amount, for example the fourth amount may be larger than the first, the second and the third amount.

It is also possible that further amounts of contrast agent are administered.

The radiological examination method may be a magnetic resonance imaging examination method and the contrast agent an MRI contrast agent. This means that the at least one input representation may be at least one MRI image and the synthetic representation may be a synthetic MRI image.

The radiological examination method may be a computed tomography examination method and the contrast agent a CT contrast agent. This means that the at least one input representation may be at least one CT image and the synthetic representation may be a synthetic CT image.

It is also possible that the at least one input representation comprises at least one MRI image and at least one CT image.

It is also possible that the radiological examination method is a computed tomography examination method and the contrast agent an MRI contrast agent.

In a preferred embodiment, the contrast agent is an MRI contrast agent (irrespective of whether it is used in a magnetic resonance tomography examination method or in a computed tomography method).

The MRI contrast agent may be an extracellular contrast agent. Extracellular contrast agents refer to low-molecular-weight, water-soluble compounds that, after intravenous administration, are distributed to the blood vessels and the interstitial spaces. After a certain, comparatively short period of circulation in the bloodstream, they are excreted via the kidneys. Extracellular MRI contrast agents include, for example, the gadolinium chelates gadobutrol (Gadovist®), gadoteridol (Prohance®), gadoteric acid (Dotarem®), gadopentetic acid (Magnevist®) and gadodiamide (Omnican®).

The MRI contrast agent may be an intracellular contrast agent. Intracellular contrast agents are taken up into the cells of tissues to a certain extent and subsequently excreted. Intracellular MRI contrast agents based on gadoxetic acid are distinguished for example in that they undergo a degree of specific uptake by liver cells (hepatocytes), accumulate in the functional tissue (parenchyma) and enhance contrasts in healthy liver tissue, before being subsequently excreted via the gallbladder into the feces. Examples of such contrast agents based on gadoxetic acid are described in U.S. Pat. No. 6,039,931A; they are commercially available for example under the trade names Primovist® and Eovist®. A further MRI contrast agent having a lower uptake into the hepatocytes is gadobenate dimeglumine (Multihance®).

Gadoxetate disodium (GD, Primovist®) belongs to the group of intracellular contrast agents. It is authorized for use in MRI of the liver for detecting and characterizing lesions in patients with known or suspected focal liver disease. With its lipophilic ethoxybenzyl unit, GD exhibits two-phase spreading: spreading at first in the intravascular and interstitial space after bolus injection, followed by selective uptake by hepatocytes. GD is excreted from the body unaltered via the kidneys and the hepatobiliary route (50:50 dual mechanism of excretion) in about the same amounts. Because of its selective accumulation in healthy liver tissue, GD is also referred to as a hepatobiliary contrast agent.

GD is approved at a dose of 0.1 ml/kg body weight (BW) (0.025 mmol/kg BW Gd). The recommended administration of GD comprises an undiluted intravenous bolus injection at a flow rate of about 2 ml/second, followed by flushing of the i.v. cannula with a physiological saline solution. A standard protocol for liver imaging using GD consists of multiple planning and pre-contrast sequences. After i.v. bolus injection of the contrast agent, dynamic images are usually acquired during the arterial phase (about 30 seconds after the injection, p.i.), portal venous phase (about 60 seconds p.i.) and transitional phase (about 2-5 minutes p.i.). Typically, the transitional phase already shows a certain rise in the liver signal intensity owing to the incipient uptake of the agent into hepatocytes. Additional T2-weighted and diffusion-weighted (DWI) images can be generated after the dynamic phase and before the late hepatobiliary phase.

In one embodiment, the contrast agent is gadoxetate disodium.

In one embodiment of the present disclosure, the contrast agent is an agent that includes gadolinium(III) 2-[4,7,10-tris(carboxymethyl)-1,4,7,10-tetrazacyclododec-1-yl]acetic acid (also referred to as gadolinium-DOTA or gadoteric acid).

In a further embodiment, the contrast agent is an agent that includes gadolinium(III) ethoxybenzyldiethylenetriaminepentaacetic acid (Gd-EOB-DTPA); preferably, the contrast agent includes the disodium salt of gadolinium(III) ethoxybenzyldiethylenetriaminepentaacetic acid (also referred to as gadoxetic acid).

In one embodiment of the present disclosure, the contrast agent is an agent that includes gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate (also referred to as gadopiclenol) (see for example WO2007/042504 and WO2020/030618 and/or WO2022/013454).

In one embodiment of the present disclosure, the contrast agent is an agent that includes dihydrogen [(±)-4-carboxy-5,8,11-tris(carboxymethyl)-1-phenyl-2-oxa-5,8,11-triazatridecan-13-oato(5-)]gadolinate(2-) (also referred to as gadobenic acid).

In one embodiment of the present disclosure, the contrast agent is an agent that includes tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate (also referred to as gadoquatrane) (see for example J. Lohrke et al.: Preclinical Profile of Gadoquatrane: A Novel Tetrameric, Macrocyclic High Relaxivity Gadolinium-Based Contrast Agent. Invest Radiol., 2022, 1, 57(10): 629-638; WO2016193190).

In one embodiment of the present disclosure, the contrast agent is an agent that includes a Gd³⁺ complex of a compound of the formula (I)

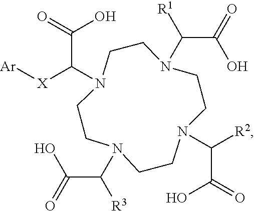

where
Ar is a group selected from

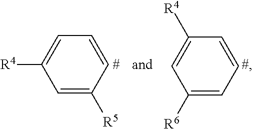

where #is the linkage to X,
X is a group selected from
CH₂, (CH₂)₂, (CH₂)₃, (CH₂)₄ and *—(CH₂)₂O—CH₂-^{#},
where * is the linkage to Ar and * is the linkage to the acetic acid residue,
R¹, R² and R³ are each independently a hydrogen atom or a group selected from C₁-C₃ alkyl, —CH₂OH, —(CH₂)₂OH and —CH₂OCH₃,
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—,
R⁵ is a hydrogen atom,
and
R⁶ is a hydrogen atom,
or a stereoisomer, tautomer, hydrate, solvate or salt thereof, or a mixture thereof.

In one embodiment of the present disclosure, the contrast agent is an agent that includes a Gd³⁺ complex of a compound of the formula (II)

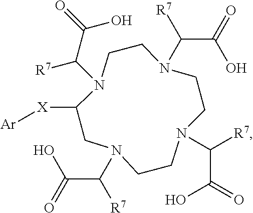

where
Ar is a group selected from

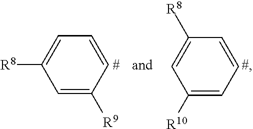

where #is the linkage to X,
X is a group selected from CH₂, (CH₂)₂, (CH₂)₃, (CH₂)₄ and *—(CH₂)₂O—CH₂-^{#}, where * is the linkage to Ar and #is the linkage to the acetic acid residue,
R⁷ is a hydrogen atom or a group selected from C₁-C₃ alkyl, —CH₂OH, —(CH₂)₂OH and —CH₂OCH₃;
R⁸ is a group selected from C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

The term “C₁-C₃ alkyl” denotes a linear or branched, saturated monovalent hydrocarbon group having 1, 2 or 3 carbon atoms, for example methyl, ethyl, n-propyl or isopropyl. The term “C₂-C₄ alkyl” denotes a linear or branched, saturated monovalent hydrocarbon group having 2, 3 or 4 carbon atoms.

The term “C₂-C₄ alkoxy” denotes a linear or branched, saturated monovalent group of the formula (C₂-C₄ alkyl)-O—, in which the term “C₂-C₄ alkyl” is as defined above, for example a methoxy, ethoxy, n-propoxy or isopropoxy group.

In one embodiment of the present disclosure, the contrast agent is an agent that includes gadolinium 2,2′,2″-(10-{1-carboxy-2-[2-(4-ethoxyphenyl)ethoxy]ethyl}-1,4,7,10-tetraazacyclododecane-1,4,7-triyl)triacetate (see for example WO2022/194777, example 1).

In one embodiment of the present disclosure, the contrast agent is an agent that includes gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 2).

In one embodiment of the present disclosure, the contrast agent is an agent that includes gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate (see for example WO2022/194777, example 4).

In one embodiment of the present disclosure, the contrast agent is an agent that includes gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate) (see for example WO2022/194777, example 15).

In one embodiment of the present disclosure, the contrast agent is an agent that includes gadolinium 2,2′,2″-{10-[(1S)-4-(4-butoxyphenyl)-1-carboxybutyl]-1,4,7,10-tetraazacyclododecane-1,4,7-triyl}triacetate (see for example WO2022/194777, example 31).

In one embodiment of the present disclosure, the contrast agent is an agent that includes gadolinium 2,2′,2″-{(2S)-10-(carboxymethyl)-2-[4-(2-ethoxyethoxy)benzyl]-1,4,7,10-tetraazacyclododecane-1,4,7-triyl}triacetate.

In one embodiment of the present disclosure, the contrast agent is an agent that includes gadolinium 2,2′,2″-[10-(carboxymethyl)-2-(4-ethoxybenzyl)-1,4,7,10-tetraazacyclododecane-1,4,7-triyl]triacetate.

In one embodiment of the present disclosure, the contrast agent is an agent that includes gadolinium(III) 5,8-bis(carboxylatomethyl)-2-[2-(methylamino)-2-oxoethyl]-10-oxo-2,5,8,11-tetraazadodecane-1-carboxylate hydrate (also referred to as gadodiamide).

In one embodiment of the present disclosure, the contrast agent is an agent that includes gadolinium(III) 2-[4-(2-hydroxypropyl)-7,10-bis(2-oxido-2-oxoethyl)-1,4,7,10-tetrazacyclododec-1-yl]acetate (also referred to as gadoteridol).

In one embodiment of the present disclosure, the contrast agent is an agent that includes gadolinium(III) 2,2′,2″-(10-((2R,3S)-1,3,4-trihydroxybutan-2-yl)-1,4,7,10-tetraazacyclododecane-1,4,7-triyl)triacetate (also referred to as gadobutrol or Gd-DO3A-butrol).

A “machine-learning model” can be understood as a computer-implemented data processing architecture. The model is able to receive input data and to supply output data on the basis of said input data and model parameters. The model can learn a relationship between the input data and the output data by means of training. During training, the model parameters can be adjusted so as to supply a desired output for a particular input.

During the training of such a model, the model is presented with training data from which it can learn. The trained machine learning model is the result of the training process. Besides input data, the training data include the correct output data (target data) that are to be generated by the model on the basis of the input data. During training, patterns that map the input data onto the target data are recognized.

In the training process, the input data of the training data are input into the model, and the model generates output data. The output data are compared with the target data. Model parameters are altered so as to reduce the differences between the output data and the target data to a (defined) minimum. The modification of model parameters in order to reduce the differences can be done using an optimization method such as a gradient descent method.

The differences can be quantified with the aid of a loss function. A loss function of this kind can be used to calculate a loss value for a given pair of output data and target data. The goal of the training process can consist in altering (adjusting) the parameters of the machine-learning model so as to reduce the loss value for all pairs of the training data set to a (defined) minimum.

For example, if the output data and the target data are numbers, the loss function can be the absolute difference between these numbers. In this case, a high absolute loss value can mean that one or more model parameters need to be altered to a substantial degree.

For example, for output data in the form of vectors, difference metrics between vectors such as the mean squared error, a cosine distance, a norm of the difference vector such as a Euclidean distance, a Chebyshev distance, an Lp norm of a difference vector, a weighted norm or any other type of difference metric between two vectors can be chosen as the loss function.

In the case of higher-dimensional outputs, such as two-dimensional, three-dimensional or higher-dimensional outputs, an element-by-element difference metric can for example be used. Alternatively or in addition, the output data may be transformed into for example a one-dimensional vector before calculation of a loss value.

In the present case, the machine-learning model is trained by means of training data to generate a synthetic representation of an examination region of an examination object on the basis of at least one input representation of the examination region of the examination object.

The training data comprise a set of input data and target data for each examination object of a multiplicity of examination objects.

The term “multiplicity” means at least ten, preferably more than one hundred.

Each set of input data and target data comprises at least one input representation of an examination region of the examination object as input data and a target representation of the examination region of the examination object and a transformed target representation of the examination region of the examination object as target data.

The examination region is normally the same for all examination objects.

Each input representation represents the examination region of the respective examination object before or after administration of a first amount of a contrast agent.

Each target representation represents the examination region of the respective examination object after the administration of a second amount of the contrast agent.

Each transformed target representation represents at least part of the examination region of the respective examination object
i) in frequency space, if the target representation represents the examination region of the examination object in real space, or
ii) in real space, if the target representation represents the examination region of the examination object in frequency space.

Each transformed target representation represents the examination region of the respective examination object after the administration of a second amount of the contrast agent.

A transformed target representation in frequency space can be generated from a target representation in real space by Fourier transform, for example.

A transformed target representation in real space can be generated from a target representation in frequency space by inverse Fourier transform, for example.

The training of the machine-learning model involves feeding, for each examination object of the multiplicity of examination objects, the at least one input representation of the examination region to the machine-learning model (i.e. inputting it into the machine-learning model). The machine-learning model generates on the basis of the at least one input representation of the examination region and on the basis of model parameters a synthetic representation.

If the at least one input representation fed to the model represents the examination region in real space, the synthetic representation preferably (but not necessarily) also represents the examination region in real space.

If the at least one input representation fed to the model represents the examination region in frequency space, the synthetic representation preferably (but not necessarily) also represents the examination region in frequency space.

The machine-learning model may be configured and trained to generate on the basis of the at least one input representation of the examination region a synthetic representation of the examination region that represents i) the examination region in frequency space, if the at least one representation represents the examination region in real space, or ii) the examination region in real space, if the at least one representation represents the examination region in frequency space. In other words: the machine-learning model may be configured and trained to carry out (inter alia) a transform from real space into frequency space or vice versa.

Generated from or in relation to the synthetic representation is a transformed synthetic representation. If the synthetic representation represents the examination region in real space, the transformed synthetic representation represents at least part of the examination region in frequency space.

If the synthetic representation represents the examination region in the second state in frequency space, the transformed synthetic representation represents at least part of the examination region in real space. The generation of the transformed synthetic representation can be achieved by transform of the synthetic representation and/or the machine-learning model may be configured and trained to generate a transformed synthetic representation on the basis of the at least one input representation.

A loss function is used to quantify the differences i) between at least part of the synthetic representation and at least part of the target representation and ii) between at least part of the transformed synthetic representation and at least part of the transformed target representation.

The loss function may comprise two terms: a first term for quantifying the differences between at least part of the synthetic representation and at least part of the target representation, and a second term for quantifying the differences between at least part of the transformed synthetic representation and at least part of the transformed target representation. The terms may, for example, be added up in the loss function. The terms may be weighted differently in the loss function. The following equation gives an example of a (total) loss function L for quantifying the differences:

L=λ₁·L₁+λ₂·L₂ (eq.1)

Here, L is the (total) loss function, L₁ is a term which represents the differences between the synthetic representation and the target representation, L₂ is a term which quantifies the differences between the transformed synthetic representation and the transformed target representation, and λ₁ and λ₂ are weight factors which, for example, can assume values between 0 and 1 and give a different weight to the two terms in the loss function. It is possible that the weight factors are kept constant or varied during the training of the machine-learning model.

Examples of loss functions that can be used to carry out the present invention are L1 loss function, L2 loss function, Lp loss function, structural similarity index measure (SSIM), VGG loss function, perceptual loss function or a combination of the aforementioned functions, or other loss functions. Further details on loss functions can be found, for example, in the scientific literature (see for example: R. Mechrez et al.: The Contextual Loss for Image Transformation with Non-Aligned Data, 2018, arXiv: 1803.02077v4; H. Zhao et al.: Loss Functions for Image Restoration with Neural Networks, 2018, arXiv:1511.08861v3; D. Fuoli et al.: Fourier Space Losses for Efficient Perceptual Image Super-Resolution, arXiv:2106.00783v1).

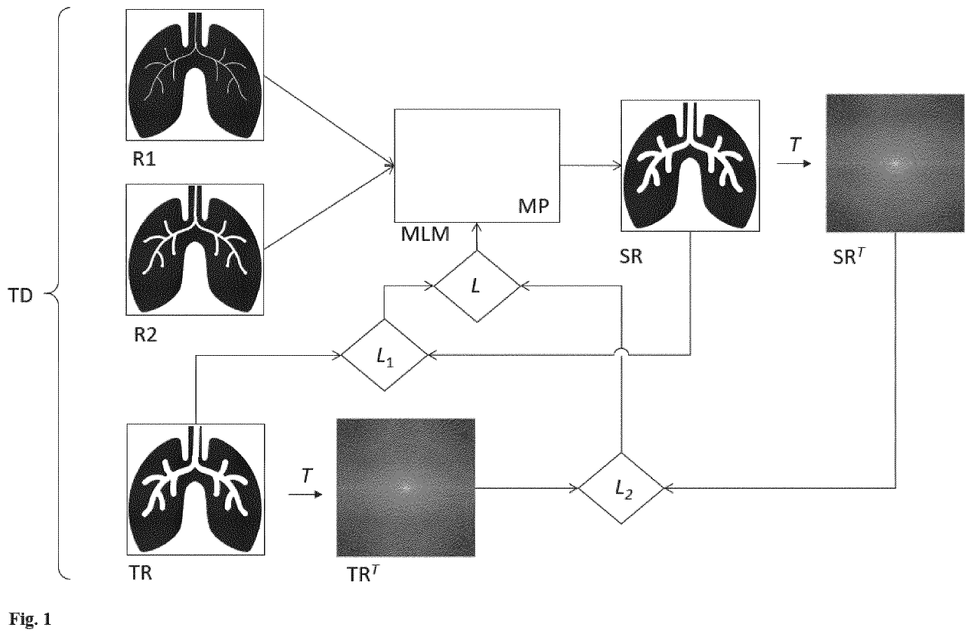

FIG. 1 shows by way of example and in schematic form the process of training the machine-learning model. The training is carried out with the aid of training data. FIG. 1 shows training data TD for an examination object. The training data TD comprise, as input data, a first input representation R1 of an examination region of the examination object and a second input representation R2 of the examination region of the examination object. The first input representation R1 and the second input representation R² represent the examination region in real space. The first input representation R1 represents the examination region, for example, without contrast agent or after the administration of a first amount of a contrast agent. The second input representation R2 represents the examination region, for example, after the administration of a second amount of the contrast agent.

The training data TD also comprise a target representation TR as target data. The target representation TR also represents the examination region in real space. The target representation TR represents the examination region, for example, after the administration of a third amount of the contrast agent.

The machine-learning model MLM is trained to predict the target representation TR on the basis of the first input representation R1 and the second input representation R2 and on the basis of model parameters MP. The first input representation R1 and the second input representation R2 are fed to the machine-learning model as input data. The machine-learning model is configured to generate a synthetic representation SR of the examination region of the examination object.

In the example shown in FIG. 1, what is generated from the synthetic representation SR by means of a transform T (e.g. a Fourier transform) is a transformed synthetic representation SR^{T}. Analogously, what is generated from the target representation TR by means of the transform T is a transformed target representation TR^{T}. The transformed synthetic representation SR^{T} and the transformed target representation TR^{T} are frequency-space depictions of the examination region after the administration of the third amount of the contrast agent.

A first loss function L₁ is used for quantifying the differences between the synthetic representation SR and the target representation TR. A second loss function L₂ is used for quantifying the differences between the transformed synthetic representation SR^{T} and the transformed target representation TR^{T}. The loss values calculated by means of the loss functions L₁ and L₂ are combined in a total loss function L (e.g. by addition with or without weighting) to give a total loss value.

Model parameters MP are modified with respect to reducing the total loss value. The total loss value can be reduced with the aid of an optimization method, for example with the aid of a gradient descent method.

The process is repeated for a multiplicity of examination objects until the total loss value has reached a predefined (desired) minimum and/or until the total loss value cannot be reduced further by modifying model parameters.

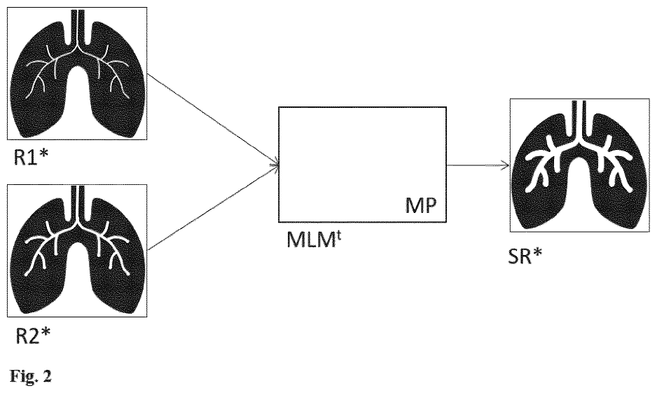

The trained machine-learning model can then be used for prediction. This is shown by way of example and in schematic form in FIG. 2: At least one input representation R1*, R2* of the examination region of a new examination object is fed to the trained machine-learning model MLM^{t}. The trained machine-learning model MLM^{t} generates a synthetic representation SR* of the examination region of the new examination object. Here, the term “new” means that input data from the new examination object have normally not already been used during the training and/or validation of the machine-learning model.

The synthetic representation SR* generated may be output, stored and/or transmitted to a separate computer system.

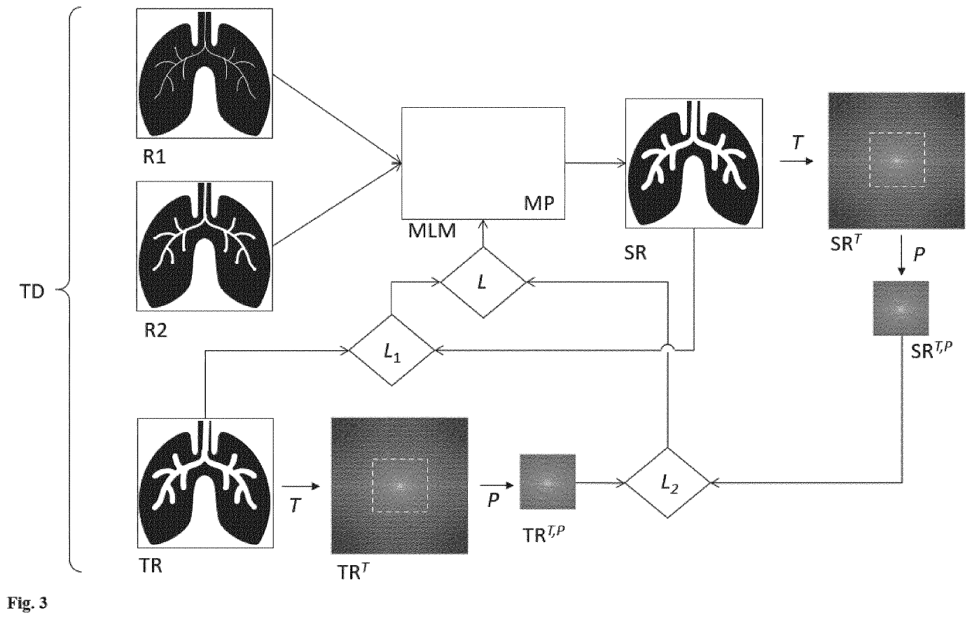

In one embodiment of the present disclosure, the quantification of the differences between the transformed synthetic representation and the transformed target representation is done only on the basis of portions of said representations. This is shown by way of example and in schematic form in FIG. 3.

The process shown in FIG. 3 corresponds to the process shown in FIG. 1, with the following differences:
The transformed target representation TR^{T} is reduced to a defined portion TR^{T,P}. The representation TR^{T,P} is part of the transformed target representation TR. The representation TR^{T,P} may be generated by a function P from the transformed target representation TR^{T}, for example by zeroing all values of frequencies outside the predefined part.
Analogously, the transformed synthetic representation SR^{T} is reduced to a defined portion SR^{T} P.
The portions to which the transformed target representation TR^{T} and the transformed synthetic representation SR^{T} are reduced are normally mutually corresponding, i.e. they normally concern the same frequencies. In the process shown in FIG. 3, both the transformed target representation TR^{T} and the transformed synthetic representation SR^{T} have each been reduced to a portion containing the low frequencies in a region (e.g. rectangle or square) around the center (see the dashed white frames). Encoded in this region is mainly contrast information.

Therefore, in the example shown in FIG. 3, the total frequency space is not considered in the calculation by means of the loss function L₂. The representations in frequency space are reduced to a region of low frequencies; the higher frequencies are discarded. Encoded in the region of low frequencies is mainly contrast information, whereas encoded in the region of higher frequencies is mainly information about fine structures. This means that, in addition to generating the synthetic representation SR, the machine-learning model in the example shown in FIG. 3 is trained to correctly reproduce in particular the low frequencies. Thus the training focuses on contrast information.

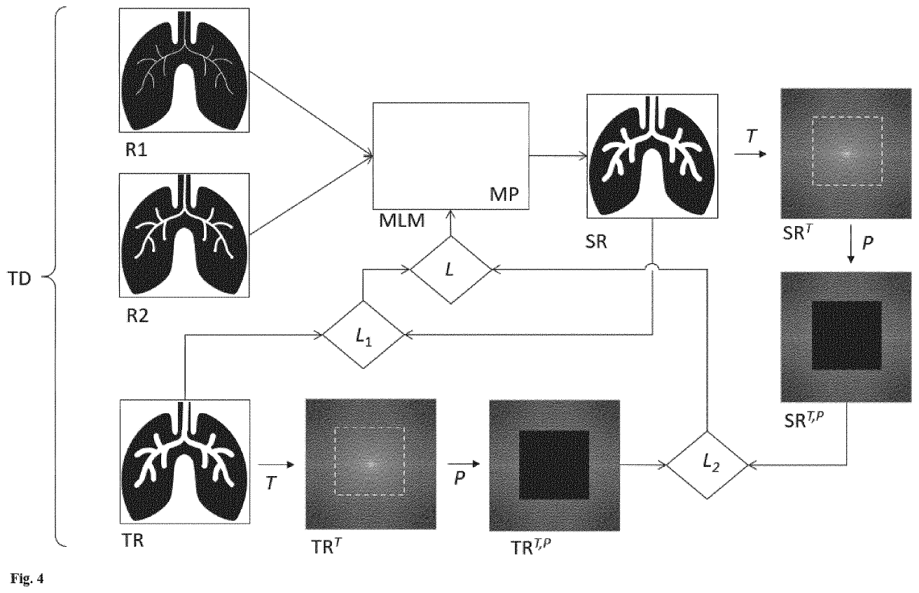

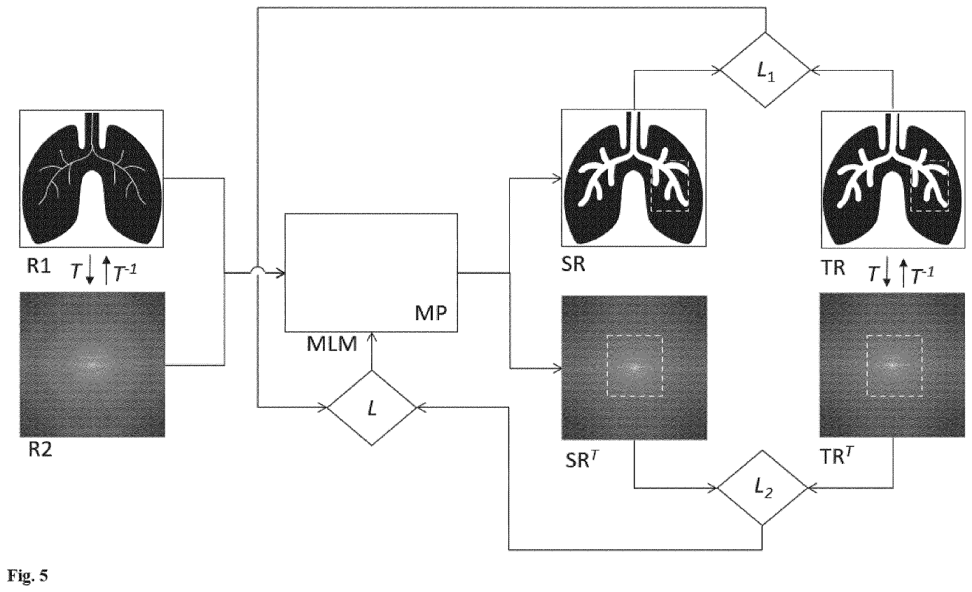

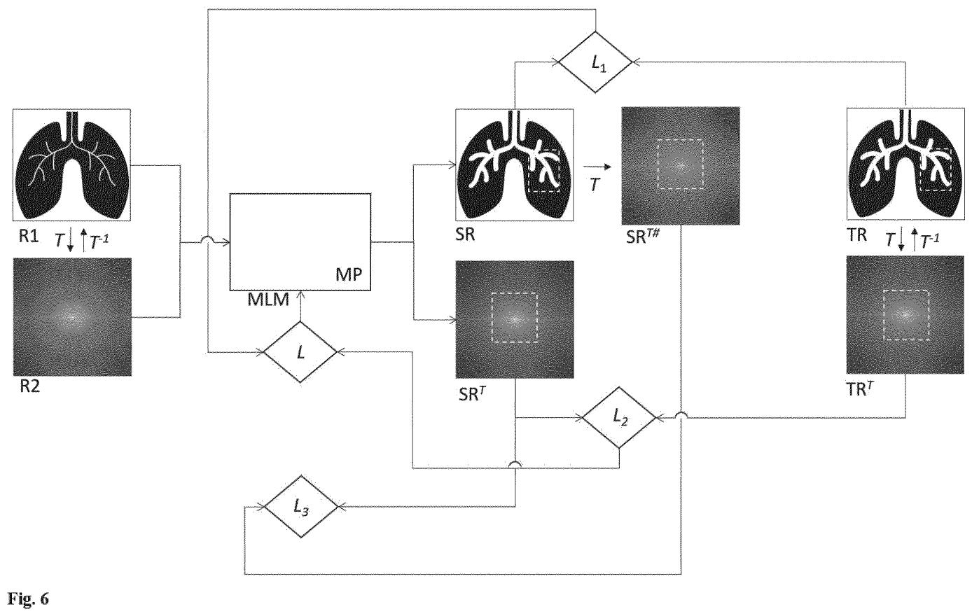

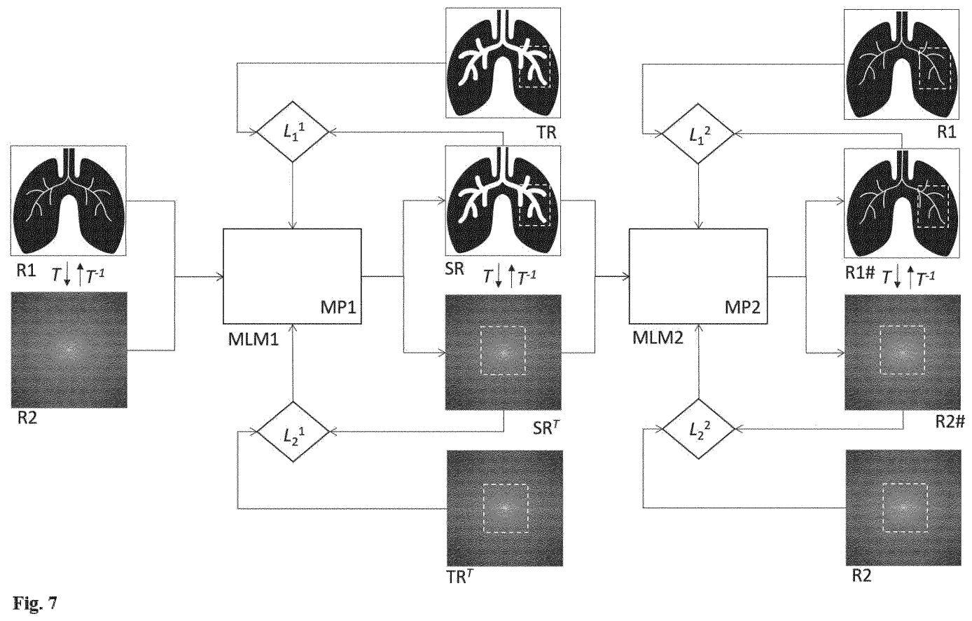

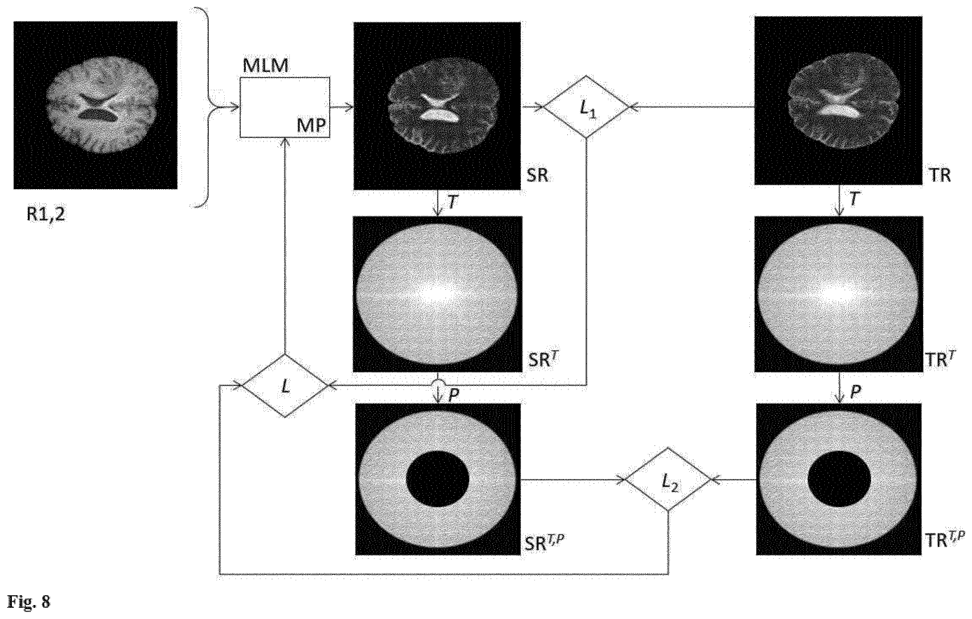

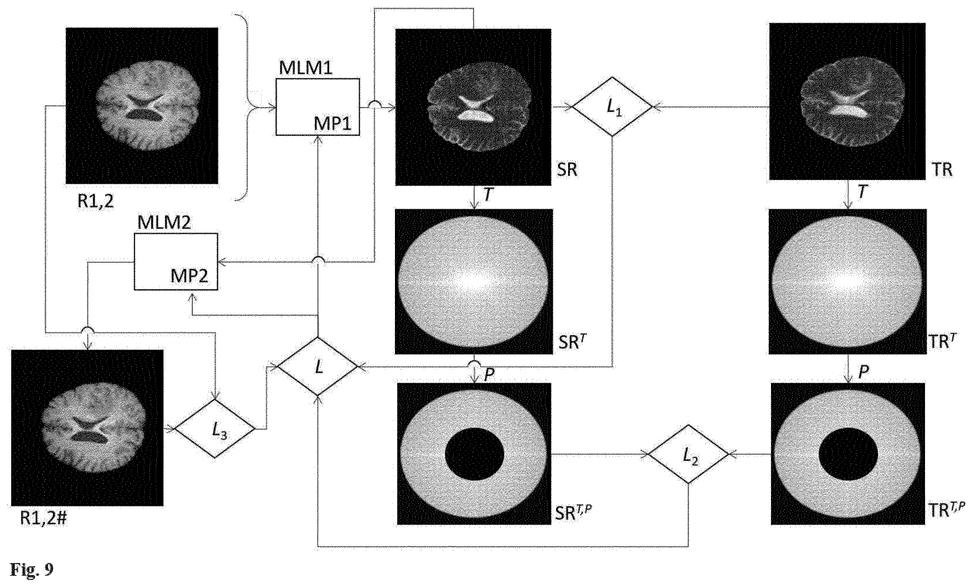

It should be noted that FIG. 3 is not to be understood as meaning that the transformed target representation TR^{T} and the transformed synthetic representation SR^{T} must be cropped to reduce them to the defined portion TR^{T,P} and the defined portion SR^{T,P}, respectively. It is also possible that the quantification of the differences between the portion SR^{T,P} and the portion TR^{T,P} by means of the loss function L₂ is achieved by only taking into account the regions SR^{T,P} and TR^{T,P} within the representations SR^{T} and TR^{T}. The term “reduce” is thus to be understood as meaning that the determination of the differences between the transformed synthetic representation SR^{T} and the transformed target representation TR^{T} is achieved by only taking into account the regions SR^{T,P} and TR^{T,P} within the representations SR^{T} and TR^{T}. This also applies analogously to FIG. 4, FIG. 5, FIG. 6, FIG. 7, FIG. 8 and FIG. 9.

FIG. 4 shows another example in which the total frequency space is not considered and in which a defined frequency range is focused on instead. The process shown in FIG. 4 corresponds to the process shown in FIG. 3, with the difference that the function P ensures that the transformed target representation TR^{T} and the transformed synthetic representation SR^{T} are each reduced to a portion comprising higher frequencies, whereas low frequencies are discarded (the low frequency values are zeroed in the present example, the value of zero being represented by the color black). This means that, in addition to generating the synthetic representation SR, the machine-learning model in the example shown in FIG. 4 is trained to correctly reproduce in particular the higher frequencies and that the focus is thus on the correct reproduction of fine structures. As already explained in relation to FIG. 3, the low frequency values do not necessarily have to be zeroed; it is also possible that the quantification of the differences between the transformed synthetic representation SR^{T} and the transformed target representation TR^{T} by means of the loss function L₂ involves only taking into account the nonblack regions. This also applies analogously to FIG. 8 and FIG. 9.

It should also be noted that the part to which the frequency space is reduced for the loss calculation may also assume shapes and sizes other than those shown in FIG. 3, FIG. 4 and FIG. 8. Furthermore, multiple parts may be selected and the frequency-space depictions may be limited (reduced) to multiple parts (regions). For example, it is possible to divide the frequency space into various regions and to generate, for multiple regions or all the regions, frequency-space depictions of the examination region that each contain only the frequencies of the respective region. This makes it possible for different frequency ranges to be weighted differently/taken into account differently during training.

In the examples shown in FIG. 3 and FIG. 4, the representations R1, R2, TR and SR are representations in real space. It is also possible that one or more of these representations or all of them are frequency-space depictions.

If the transformed synthetic representation SR^{T} and the transformed target representation TR^{T} are representations in real space, a part (or multiple parts) in the representations may likewise be selected and the representations may be reduced to said part(s). In such a case, the loss calculation thus focuses on features in real space that are found in the selected part(s).

FIG. 5, FIG. 6, FIG. 7, FIG. 8 and FIG. 9 show further embodiments of the training method of the present disclosure.

In the embodiment shown in FIG. 5, the training data comprise for each examination object at least one input representation R1 of an examination region and a target representation TR of the examination region. The at least one input representation R1 represents the examination region without and/or after administration of a first amount of a contrast agent. The synthetic representation SR and the target representation TR represent the examination region after the administration of a second amount of the contrast agent.

The at least one input representation R1 and the target representation TR may each be a representation in real space or a representation in frequency space. In the example shown in FIG. 5, the input representation R1 and the target representation TR are representations in real space. The machine-learning model MLM is trained to predict the target representation TR on the basis of the at least one input representation R1. A transform T is used to generate a transformed input representation R2 on the basis of the input representation R1. Analogously, the transform T is used to generate a transformed target representation TR^{T} on the basis of the target representation TR. In the example shown in FIG. 5, the transformed input representation R2 and the transformed target representation TR^{T} are representations of the examination region in frequency space. The transformed input representation R2 represents the examination region (such as the input representation R1) without and/or after the administration of the first amount of a contrast agent; the transformed target representation TR^{T} represents the examination region (such as the target representation TR) after the administration of the second amount of the contrast agent.

FIG. 5 shows that the input representation R1 can be obtained by an inverse transform T¹ on the basis of the transformed input representation R2. Analogously, a target representation TR can be obtained by the inverse transform T¹ on the basis of the transformed target representation TR^{T}. The inverse transform T¹ is the inverse transform in relation to the transform T, and this is indicated by the superscript index −1. The reference to the inverse transform is intended to make it clear that the training data must contain at least an input representation (R1 or R2) and a target representation (TR or TR^{T}); the other input representation (R2 or R1) and the other target representation (TR^{T} or TR) can be obtained from the existing representation by transform or inverse transform. This is generally applicable and does not only apply to the embodiment shown in FIG. 5.

In the embodiment shown in FIG. 5, both the input representation R1 and the transformed input representation R2 are fed to the machine-learning model MLM. The machine-learning model MLM is configured to generate both a synthetic representation SR and a transformed synthetic representation SR^{T}.

A first loss function L₁ is used to quantify the differences between at least part of the synthetic representation SR and at least part of the target representation TR. A second loss function L₂ is used to quantify the differences between at least part of the transformed synthetic representation SR^{T} and at least part of the transformed target representation TR^{T}. The dashed white frames in the transformed representations in FIG. 5 are intended to express that the loss calculation by means of the loss function does not have to be based on the entire frequency-space depiction, as described in relation to FIG. 3 and FIG. 4, and that, instead, the transformed representations can be reduced to a frequency range (or multiple frequency ranges). Analogously, the real-space depictions SR and TR can be reduced to one part (or multiple parts) for loss calculation (also shown by the dashed white frames). This also applies analogously to the other embodiments and does not only apply to the embodiment shown in FIG. 5.

The loss values calculated by means of the loss functions L₁ and L₂ are combined in a total loss function L (e.g. by addition with or without weighting) to give a total loss value.

Model parameters MP are modified with respect to reducing the total loss value. This can be done by means of an optimization method, for example a gradient descent method.

The process is repeated for a multiplicity of examination objects until the total loss value has reached a predefined (desired) minimum and/or until the total loss value can no longer be reduced by modifying model parameters.

The synthetic representation SR and the transformed synthetic representation SR^{T} must be interconvertible just like the target representation TR and the transformed target representation TR^{T}. It is possible to introduce a further loss function that assesses the quality of such a conversion. It is thus possible to generate a transformed synthetic representation SR^{T} from the synthetic representation SR by the transform T and to quantify the differences between this transformed synthetic representation generated by transform and the transformed synthetic representation generated by the machine-learning model by means of a third loss function L₃. Alternatively or additionally, it is possible to generate a synthetic representation SR from the transformed synthetic representation SR^{T} by the inverse transform T¹ and to quantify the differences between this synthetic representation generated by inverse transform and the synthetic representation generated by the machine-learning model by means of a third loss function L₃. This is shown schematically in FIG. 6 using the example of generating a transformed synthetic representation SR^{T #}from the synthetic representation SR by transform T. In the example shown in FIG. 6, the loss function L₃ quantifies the differences between the transformed synthetic representation SR^{T #}generated by transform and the transformed synthetic representation SR^{T} generated by the machine-learning model MLM. The loss functions L₁, L₂ and L₃ are combined in a total loss function L (e.g. by addition with or without weighting). In the total loss function L, the individual terms may have different weights, and the weights may be kept constant or varied over the course of the training.

A modification of the embodiments shown in FIG. 5 and FIG. 6 is that the machine-learning model MLM is not fed the two input representations R1 and R2, but just one of the two representations. The machine-learning model may be configured and trained to generate the other of the two representations.

FIG. 7 shows in schematic form a further embodiment of the training method. In this embodiment, two machine-learning models are used: a first model MLM1 and a second model MLM2. The first machine-learning model MLM1 is trained to predict a target representation TR and/or a transformed target representation TR^{T} from an input representation R1 and/or R2. The second machine-learning model MLM2 is trained to predict the original input representation R1 and/or R2 again (to reconstruct it/them) from a predicted target representation SR and/or a predicted transformed target representation SR^{T}. The machine-learning models thus perform a cycle which can improve the quality of prediction by the first model MLM1 (which is used for later prediction).

The first machine-learning model MLM1 is fed at least one input representation R1 and/or at least one transformed input representation R2 as input data. The first model MLM1 is configured to generate a synthetic representation SR and/or a transformed synthetic representation SR^{T} on the basis of the input data and model parameters MP1. A transformed synthetic representation SR^{T} may also be generated by transform T of the synthetic representation SR. A synthetic representation SR may also be generated by inverse transform T¹ of the transformed synthetic representation SR^{T}.

Differences between the synthetic representation SR and the target representation TR may be quantified by means of a loss function L₁¹. Differences between the transformed synthetic representation SR^{T} and the transformed target representation TR^{T} may be quantified by means of a loss function L₂¹.

Differences between a synthetic representation SR obtained by inverse transform T¹ and the synthetic representation SR generated by the model MLM1 may be quantified by means of a loss function L₃¹ (not shown in FIG. 7). Alternatively or additionally, differences between a transformed synthetic representation SR^{T} obtained by transform T and the transformed synthetic representation SR^{T} generated by the model MLM1 may be quantified by means of a loss function L₄¹ (not shown in FIG. 7).

The loss functions L₁¹, L₂¹ and, if present, L₃¹ and/or L₄¹ may be combined to form a total loss function (not shown in FIG. 7).

Model parameters MP1 may be modified with respect to reducing the total loss value.

The synthetic representation SR and/or the transformed synthetic representation SR^{T} is/are fed to the second machine-learning model MLM2. The second model MLM2 is configured to reconstruct (predict) the first input representation R1 and/or the second input representation R2. The second model MLM2 is configured to generate a predicted first input representation R1 #and/or a predicted second input representation R2 #on the basis of the synthetic representation SR and/or the transformed synthetic representation SR^{T} and on the basis of model parameters MP2. A second input representation R2 #may also be generated by transform T of the first input representation R1 #and/or a first input representation R1 #may also be generated by inverse transform T¹ of the second input representation R2 #.

Differences between the predicted first input representation R1 #and the first input representation R1 may be quantified by means of a loss function L₁². Differences between the predicted second input representation R2 #and the second input representation R2 may be quantified by means of a loss function L2.

Differences between an input representation obtained by inverse transform T¹ and the input representation generated by the model MLM2 may be quantified by means of a loss function L32 (not shown in FIG. 7). Alternatively or additionally, differences between an input representation obtained by transform and the input representation generated by the model MLM2 may be quantified by means of a loss function L42 (not shown in FIG. 6).

The loss functions L₁², L₂² and, if present, L₃² and/or L₄² may be combined to form a total loss function (not shown in FIG. 7).

Model parameters MP2 may be modified with respect to reducing the total loss value.

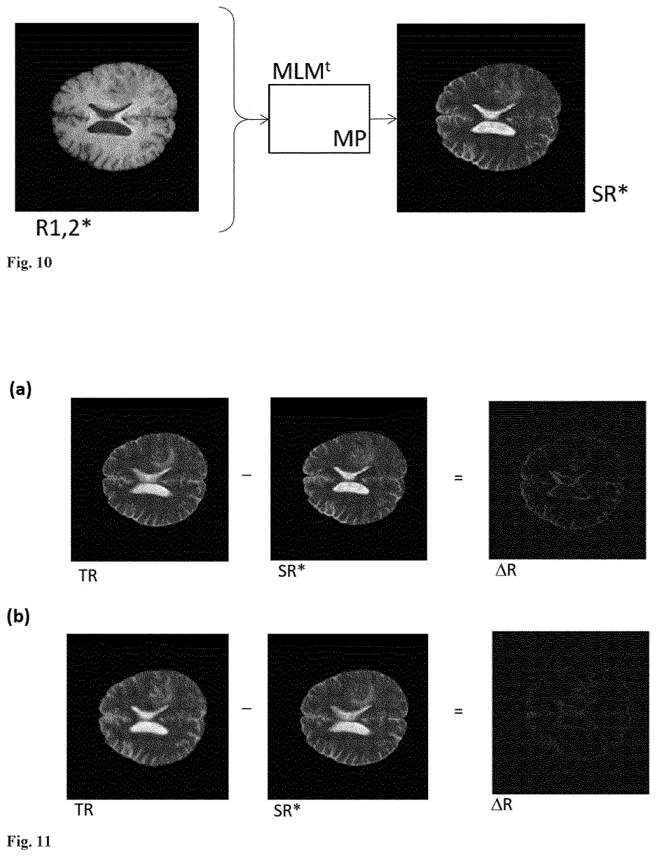

FIG. 8 and FIG. 9 show schematically further examples of the method for training the machine-learning model. FIG. 10 shows schematically a further example of the use of the trained machine-learning model for prediction.

In the example shown in FIG. 8, the machine-learning model MLM is trained to generate, on the basis of one or more input representation(s) R1,2 representing an examination region of an examination object before and/or after the administration of a first amount of a contrast agent and on the basis of model parameters MP, a synthetic representation SR of the examination region of the examination object, the synthetic representation SR representing the examination region after the administration of a second amount of the contrast agent, the second amount being larger than the first amount. In other words, the machine-learning model is trained to predict a representation of the examination region after the administration of an amount of contrast agent larger than the amount actually administered. A target representation TR represents the examination region of the examination object after the administration of the second (larger) amount of the contrast agent.

In the present example, the examination region is a brain of a human. The at least one input representation R1,2, the target representation TR and the synthetic representation SR represent the brain in real space.

The at least one input representation R1,2 is input into the machine-learning model MLM. The machine-learning model generates the synthetic representation SR.

A transform T is used to transform the synthetic representation SR into a transformed synthetic transformation SR^{T}. The transformed synthetic transformation SR^{T} is reduced with the aid of a function P to one part; what is formed is a partial transformed synthetic transformation SR^{T}.

Analogously, generated from the target representation SR by means of the transform T is a transformed target representation TR^{T}, and generated by means of the function P from the transformed target representation TR^{T} is a partial transformed target representation TR^{T,P}.

The transformed synthetic representation SR^{T} and the transformed target representation TR^{T} represent the examination region in frequency space. The partial transformed synthetic representation SR^{T,P} is a transformed synthetic representation SR^{T} reduced to a frequency range of higher frequencies. The partial transformed target representation TR^{T,P} is a transformed target representation TR^{T} reduced to the frequency range of higher frequencies. The machine-learning model MLM is thus trained to focus on fine structure information encoded in the higher frequencies.

The machine-learning model is trained with the aid of a loss function L. The loss function L quantifies the differences i) between the synthetic representation SR and the target representation TR by means of a first term L₁ and ii) between the partial transformed synthetic representation SR^{T,P} and the partial transformed target representation TR^{T,P} by means of a second term L₂. In the loss function, the first term L₁ and the second term L₂ may, for example, each be multiplied by a weight factor followed by addition of the resulting products (as described by the equation eq. 1).

In an optimization method, for example a gradient descent method, the model parameters MP can be modified with respect to reducing the loss value calculated by means of the function L.

The process described is repeated for other examination objects. The training can be ended when the loss values calculated by means of the loss function L reach a defined minimum, i.e. the quality of prediction reaches a desired level.

FIG. 9 shows the training method discussed in relation to FIG. 8, having the following augmentations/changes:
In the training method, two machine-learning models are used: a first model MLM1 and a second model MLM2.
The first machine-learning model MLM1 executes the functions described in relation to FIG. 8 for the machine-learning model MLM.
The second machine-learning model MLM2 is configured and trained to reproduce the at least one input representation R1,2. In other words, the second machine-learning model MLM2 is configured and trained to generate a predicted input representation R1,2 #on the basis of the synthetic representation SR and on the basis of model parameters MP2.
The loss function L has a third term L₃ which quantifies the differences between the input representation R1,2 and the predicted input representation R1,2 #.
In an optimization method, for example a gradient descent method, the model parameters MP1 and MP2 can be modified with respect to reducing the loss value calculated by means of the function L.

FIG. 10 shows the use of the machine-learning model MLM or MLM1 trained according to FIG. 8 or FIG. 9, the trained model being identified by MLM^{t} in FIG. 10.

The trained machine-learning model MLM^{t} is fed at least one input representation R1,2* representing the brain of a new examination object. The term “new” means that the at least one input representation R1,2* has not already been used in training. The at least one input representation R1,2* represents the brain of the new examination object before and/or after the administration of a first amount of a contrast agent.

The trained machine-learning model generates, on the basis of the at least one input representation R1,2*, a synthetic representation SR* representing the brain after the administration of a second amount of the contrast agent.

FIG. 11 shows the result of validation (a) of the machine-learning model trained according to FIG. 8 and (b) of the machine-learning model trained according to FIG. 9. Each of the models was fed at least one input representation of a new examination object, described in relation to FIG. 10. Each of the models respectively generates a synthetic representation SR*. A target representation TR exists for the examination object. FIG. 11 shows the generation of a differential representation; for each model, a differential representation AR is generated by subtraction of the respective synthetic representation SR* from the target representation TR. In the case of a perfect model, each of the synthetic representations SR* corresponds to the target representation TR and the differential representation AR is completely black (all values are zero). It can be seen in FIG. 11 that, in the case of the machine-learning model trained according to FIG. 8 (FIG. 11 (a)), more pixels differ from zero than in the case of the machine-learning model trained according to FIG. 9 (FIG. 11 (b)). Thus, in the present example, the quality of prediction by the machine-learning model trained according to FIG. 9 is higher than the quality of prediction by the machine-learning model trained according to FIG. 8.

The machine-learning models according to the present disclosure may, for example, be an artificial neural network or include such an artificial neural network or a plurality thereof.

An artificial neural network comprises at least three layers of processing elements: a first layer with input neurons (nodes), an N-th layer with at least one output neuron (nodes), and N-2 inner layers, where N is a natural number and greater than 2.

The input neurons serve to receive the at least one input representation. Normally, there is one input neuron for each pixel or voxel of an input representation when the representation is a real-space depiction in the form of a raster graphic, or one input neuron for each frequency present in the input representation when the representation is a frequency-space depiction. There may be additional input neurons for additional input values (for example information about the examination region, information about the examination object, information about conditions when the input representation was generated, information about the state represented by the input representation, information about the moment at which or period of time in which the input representation has been generated, and/or information about the amount of contrast agent administered).

The output neurons can serve to output a synthetic representation.

The processing elements of the layers between the input neurons and the output neurons are connected to one another in a predetermined pattern with predetermined connection weights.

Preferably, the artificial neural network is a so-called convolutional neural network (CNN) or comprises such a network.

A CNN normally consists essentially of an alternately repeating array of filters (convolutional layer) and aggregation layers (pooling layer) terminating in one or more layers of “normal” fully connected neurons (dense/fully connected layer).

The training of the neural network may, for example, be carried out by means of a back propagation method. The goal for the network is to map the at least one input representation onto the synthetic representation as reliably as possible. The quality of prediction is described by a loss function. The goal is to minimize the loss function. In the case of the back propagation method, an artificial neural network is taught by altering the connection weights.

In the trained state, the connection weights between the processing elements contain information regarding the dynamics of the relationship between the input representation(s) and the synthetic representation that can be used to predict, on the basis of one or more representations of the examination region of a new examination object, a synthetic representation of the examination region of the new examination object. Here, the term “new” means that representations of the examination region of the new examination object have not already been used during the training of the machine-learning model.

A cross-validation method may be used to divide the data into training data sets and validation data sets.

The training data set is used in the back propagation training of network weights. The validation data set is used to check the accuracy of prediction with which the trained network can be applied to unknown (new) data.

The artificial neural network may have an autoencoder architecture; for example the artificial neural network may have an architecture such as the U-Net (see for example O. Ronneberger et al.: U-net: Convolutional networks for biomedical image segmentation, International Conference on Medical image computing and computer-assisted intervention, pages 234-241, Springer, 2015, https://doi.org/10.1007/978-3-319-24574-4_28).

The artificial neural network may be a generative adversarial network (GAN) (see for example M. Y. Liu et al.: Generative Adversarial Networks for Image and Video Synthesis: Algorithms and Applications, arXiv:2008.02793; J. Henry et al.: Pix2Pix GAN for Image-to-Image Translation, DOI: 10.13140/RG.2.2.32286.66887).

The artificial neural network may be a regularized generative adversarial network (see for example Q. Li et al.: RegGAN: An End-to-End Network for Building Footprint Generation with Boundary Regularization, Remote Sens. 2022, 14, 1835).

The artificial neural network may be a conditional adversarial network (see for example P. Isola et al.: Image-to-Image Translation with Conditional Adversarial Networks, arXiv:1611.07004 [cs.CV]).

The artificial neural network may be a transformer network (see for example D. Karimi et al.: Convolution-Free Medical Image Segmentation using Transformers, arXiv:2102.13645 [eess.IV]).

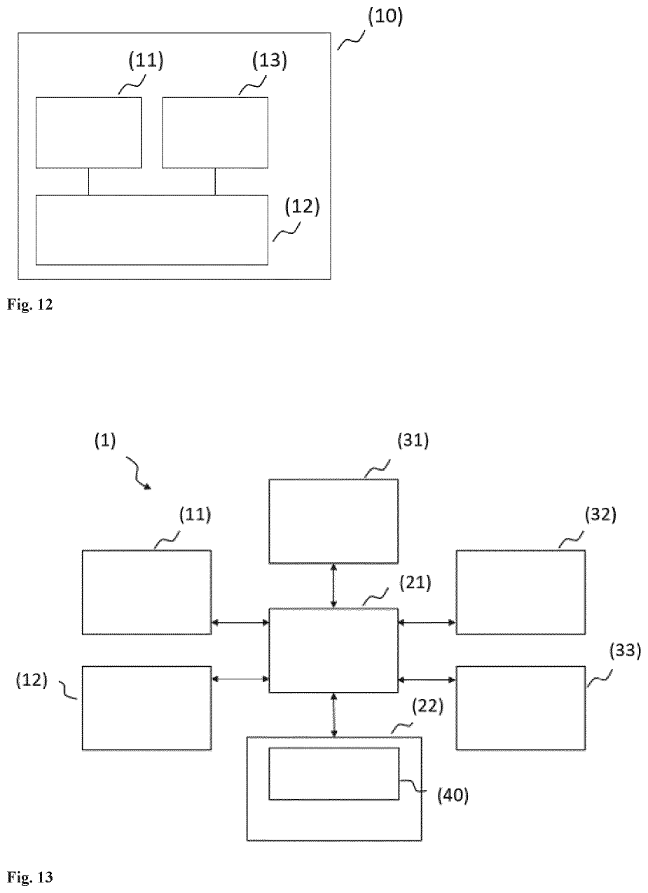

FIG. 12 shows by way of example and in schematic form a computer system according to the present disclosure, which can be used for training the machine-learning model and/or for using the trained machine-learning model for prediction.

A “computer system” is an electronic data processing system that processes data by means of programmable calculation rules. Such a system typically comprises a “computer”, which is the unit that includes a processor for carrying out logic operations, and peripherals.

In computer technology, “peripherals” refers to all devices that are connected to the computer and are used for control of the computer and/or as input and output devices. Examples thereof are monitor (screen), printer, scanner, mouse, keyboard, drives, camera, microphone, speakers, etc. Internal ports and expansion cards are also regarded as peripherals in computer technology.

The computer system (10) shown in FIG. 12 comprises a receiving unit (11), a control and calculation unit (12) and an output unit (13).

The control and calculation unit (12) serves for control of the computer system (10), for coordination of the data flows between the units of the computer system (10), and for the performance of calculations.

The control and calculation unit (12) is configured:
to provide a trained machine-learning model,
to cause the receiving unit (11) to receive at least one input representation of an examination region of an examination object, the at least one input representation representing the examination region without and/or after administration of a first amount of a contrast agent,
to input the received at least one input representation into the trained machine-learning model,
to receive from the machine-learning model a synthetic representation of the examination region of the examination object,
to cause the output unit (13) to output the synthetic representation of the examination region of the examination object and/or to store it and/or to transmit it to a separate computer system.

FIG. 13 shows by way of example and in schematic form a further embodiment of the computer system according to the invention. The computer system (1) comprises a processing unit (21) connected to a memory (22). The processing unit (21) and the memory (22) form a control and calculation unit, as shown in FIG. 12.

The processing unit (21) may comprise one or more processors alone or in combination with one or more memories. The processing unit (21) may be customary computer hardware that is able to process information such as digital images, computer programs and/or other digital information. The processing unit (21) normally consists of an arrangement of electronic circuits, some of which can be designed as an integrated circuit or as a plurality of integrated circuits connected to one another (an integrated circuit is sometimes also referred to as a “chip”). The processing unit (21) may be configured to execute computer programs that can be stored in a working memory of the processing unit (21) or in the memory (22) of the same or of a different computer system.

The memory (22) may be customary computer hardware that is able to store information such as digital images (for example representations of the examination region), data, computer programs and/or other digital information either temporarily and/or permanently. The memory (22) may comprise a volatile and/or nonvolatile memory and may be nonremovable or removable. Examples of suitable memories are RAM (random access memory), ROM (read-only memory), a hard disk, a flash memory, an exchangeable computer floppy disk, an optical disc, a magnetic tape or a combination of the aforementioned. Optical discs can include compact discs with read-only memory (CD-ROM), compact discs with read/write function (CD-R/W), DVDs, Blu-ray discs and the like.

The processing unit (21) may be connected not just to the memory (22), but also to one or more interfaces (11, 12, 31, 32, 33) in order to display, transmit and/or receive information. The interfaces may comprise one or more communication interfaces (32, 33) and/or one or more user interfaces (11, 12, 31). The one or more communication interfaces may be configured to send and/or receive information, for example to and/or from an MRI scanner, a CT scanner, an ultrasound camera, other computer systems, networks, data memories or the like. The one or more communication interfaces may be configured to transmit and/or receive information via physical (wired) and/or wireless communication connections. The one or more communication interfaces may comprise one or more interfaces for connection to a network, for example using technologies such as cellphone, Wi-Fi, satellite, cable, DSL, optical fiber and/or the like. In some examples, the one or more communication interfaces may comprise one or more close-range communication interfaces configured to connect devices with close-range communication technologies such as NFC, RFID, Bluetooth, Bluetooth LE, ZigBee, infrared (e.g. IrDA) or the like.

The user interfaces may include a display (31). A display (31) may be configured to display information to a user. Suitable examples thereof are a liquid crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces are a microphone, an image or video recording device (for example a camera), a keyboard or a keypad, a joystick, a touch-sensitive surface (separate from a touchscreen or integrated therein) or the like. In some examples, the user interfaces may contain an automatic identification and data capture technology (AIDC) for machine-readable information. This can include barcodes, radiofrequency identification (RFID), magnetic strips, optical character recognition (OCR), integrated circuit cards (ICC) and the like.

The user interfaces may furthermore include one or more interfaces for communication with peripherals such as printers and the like.

One or more computer programs (40) may be stored in the memory (22) and executed by the processing unit (21), which is thereby programmed to perform the functions described in this description. The retrieving, loading and execution of instructions of the computer program (40) may take place sequentially, such that an instruction is respectively retrieved, loaded and executed. However, the retrieving, loading and/or execution may also take place in parallel.

The machine-learning model according to the invention may also be stored in the memory (22).

The computer system according to the invention may be designed as a laptop, notebook, netbook and/or tablet PC; it may also be a component of an MRI scanner, a CT scanner or an ultrasound diagnostic device.

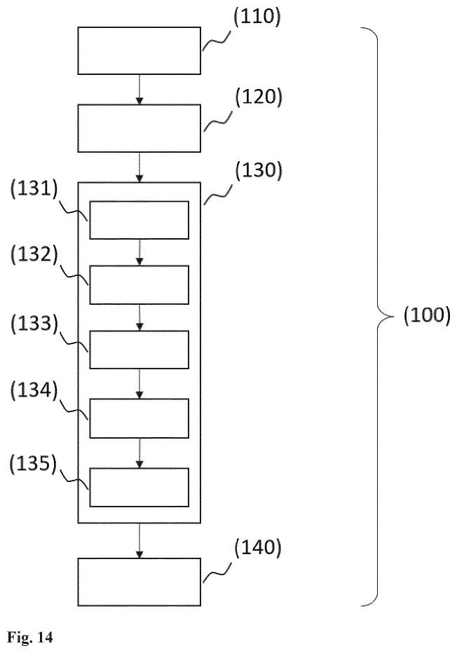

FIG. 14 shows schematically in the form of a flow chart one embodiment of the method for training a machine-learning model. The training method (100) comprises the steps of:
(110) receiving and/or providing training data, the training data comprising a set of input data and target data for each examination object of a multiplicity of examination objects,
   each set comprising at least one input representation of an examination region of the examination object as input data and a target representation of the examination region of the examination object and a transformed target representation as target data,
   the at least one input representation representing the examination region without and/or after administration of a first amount of a contrast agent and the synthetic representation representing the examination region after the administration of a second amount of the contrast agent,
   the transformed target representation representing at least part of the examination region of the examination object
      in frequency space, if the target representation represents the examination region of the examination object in real space, or
      in real space, if the target representation represents the examination region of the examination object in frequency space,
(120) receiving and/or providing a machine-learning model, the machine-learning model being configured to generate on the basis of at least one input representation of an examination region of an examination object and model parameters a synthetic representation of the examination region of the examination object,
(130) training the machine-learning model, the training comprising for each examination object of the multiplicity of examination objects:
   (131) feeding the at least one input representation to the machine-learning model,
   (132) receiving a synthetic representation of the examination region of the examination object from the machine-learning model,
   (133) generating and/or receiving a transformed synthetic representation on the basis of the synthetic representation and/or in relation to the synthetic representation, the transformed synthetic representation representing at least part of the examination region of the examination object
      in frequency space, if the synthetic representation represents the examination region of the examination object in real space, or
      in real space, if the synthetic representation represents the examination region of the examination object in frequency space,
   (134) quantifying the differences i) between at least part of the synthetic representation and at least part of the target representation and ii) between at least part of the transformed synthetic representation and at least part of the transformed target representation by means of a loss function,
   (135) reducing the differences by modifying model parameters,
(140) outputting and/or storing the trained machine-learning model and/or the model parameters and/or transmitting the trained machine-learning model and/or the model parameters to a separate computer system and/or using the trained machine-learning model for generation of a synthetic radiological image for a new examination object.

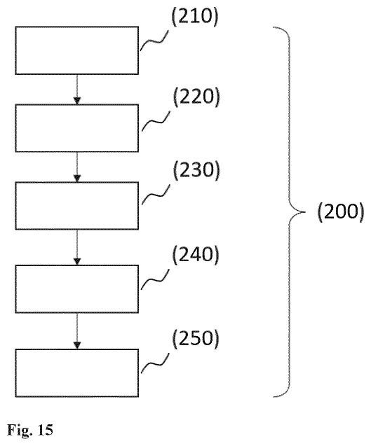

FIG. 15 shows schematically in the form of a flow chart one embodiment of the method for generating a synthetic representation of an examination region of anew examination object with the aid of the trained machine-learning model. The prediction method (200) comprises the steps of
(210) providing a trained machine-learning model,
   the trained machine-learning model having been trained by means of training data to generate on the basis of at least one input representation of an examination region of an examination object a synthetic representation of the examination region of the examination object, the at least one input representation representing the examination region without and/or after administration of a first amount of a contrast agent and the synthetic representation representing the examination region after the administration of a second amount of the contrast agent,
   the training data comprising for each examination object of a multiplicity of examination objects i) at least one input representation of the examination region, ii) a target representation of the examination region and iii) a transformed target representation of the examination region,
   the transformed target representation representing at least part of the examination region in frequency space, if the target representation represents the examination region in real space, or in real space, if the target representation represents the examination region in frequency space,
   the training of the machine-learning model comprising reducing differences between i) at least part of the synthetic representation and at least part of the target representation and ii) between at least part of a transformed synthetic representation and at least part of the transformed target representation,
(220) receiving at least one input representation of an examination region of anew examination object, the at least one input representation representing the examination region of the new examination object without and/or after administration of a first amount of a contrast agent,
(230) inputting the at least one input representation of the examination region of the new examination object into the trained machine-learning model,
(240) receiving a synthetic representation of the examination region of the new examination object from the machine-learning model,
(250) outputting and/or storing the synthetic representation of the examination region of the new examination object and/or transmitting the synthetic representation of the examination region of the new examination object to a separate computer system.

1. A computer-implemented method comprising:
providing a trained machine-learning model (MLM^{t});
the trained machine-learning model (MLM^{t}) having been trained by means of training data (TD) to generate on the basis of at least one input representation (R1, R2) of an examination region of an examination object a synthetic representation (SR) of the examination region of the examination object; and
the training data (TD) comprising for each examination object of a multiplicity of examination objects i) an input representation (R1, R2) of the examination region of the examination object, ii) a target representation (TR) of the examination region of the examination object and iii) a transformed target representation (TR^{T});
the at least one input representation (R1, R2) representing the examination region of the respective examination object without or after administration of a first amount of a contrast agent;
the target representation (TR) representing the examination region of the respective examination object after administration of a second amount of the contrast agent; and
the transformed target representation (TR^{T}) representing at least part of the examination region of the examination object in frequency space, if the target representation (TR) represents the examination region of the examination object in real space, or in real space, if the target representation (TR) represents the examination region of the examination object in frequency space;
the training of the machine-learning model (MLM1) comprising reducing the differences between i) at least part of the synthetic representation (SR) and at least part of the target representation (TR) and ii) between at least part of a transformed synthetic representation (SR^{T}) and at least part of the transformed target representation (TR^{T});
receiving at least one input representation (R1*, R2*) of an examination region of a new examination object, the at least one input representation (R1*, R2*) representing the examination region of the new examination object without the contrast agent and/or after administration of a first amount of the contrast agent;
inputting the at least one input representation (R1*, R2*) of the examination region of the new examination object into the trained machine-learning model (MLM^{t});
receiving a synthetic representation (SR*) of the examination region of the new examination object from the trained machine-learning model (MLM); and
outputting and/or storing the synthetic representation (SR*) of the examination region of the new examination object and/or transmitting the synthetic representation (SR*) of the examination region of the new examination object to a separate computer system.

2. The method as claimed in claim 1, wherein the training of the machine-learning model (MLM^{t}) comprises:
receiving and/or providing the training data (TD), the training data (TD) comprising a set of input data and target data for each examination object of the multiplicity of examination objects;
each set comprising the at least one input representation (R1, R2) of the examination region of the examination object as input data and the target representation (TR) of the examination region of the examination object and the transformed target representation (TR^{T}) as target data;
the at least one input representation (R1, R2) representing the examination region without contrast agent and/or after administration of the first amount of the contrast agent and the target representation (TR) representing the examination region after the administration of the second amount of the contrast agent; and
the transformed target representation (TR^{T}) representing at least part of the examination region of the examination object;
in frequency space, if the target representation (TR) represents the examination region of the examination object in real space; or
in real space, if the target representation (TR) represents the examination region of the examination object in frequency space;
receiving and/or providing a machine-learning model (MLM), the machine-learning model (MLM) being configured to generate on the basis of at least one input representation (R1, R2) of an examination region of an examination object and model parameters (MP) a synthetic representation (SR) of the examination region of the examination object; and
training the machine-learning model (MLM), the training comprising for each examination object of the multiplicity of examination object;
feeding the at least one input representation (R1, R2) of the examination region of the examination object to the machine-learning model (MLM);
receiving a synthetic representation (SR) of the examination region of the examination object from the machine-learning model (MLM); and
generating and/or receiving a transformed synthetic representation (SR^{T}) on the basis of the synthetic representation (SR) and/or in relation to the synthetic representation (SR), the transformed synthetic representation (SR^{T}) representing at least part of the examination region of the examination object;
in frequency space, if the synthetic representation (SR) represents the examination region of the examination object in real space; or
in real space, if the synthetic representation (SR) represents the examination region of the examination object in frequency space;
quantifying the differences i) between at least part of the synthetic representation (SR) and at least part of the target representation (TR) and ii) between at least part of the transformed synthetic representation (SR^{T}) and at least part of the transformed target representation (TR^{T}) by means of a loss function (L); and
reducing the differences by modifying model parameters (MP);
outputting and/or storing the trained machine-learning model (MLM) and/or transmitting the trained machine-learning model (MLM) to a separate computer system.

3. The method as claimed in claim 2,
wherein the receiving and/or providing of the training data (TD) comprises:
generating a partial transformed target representation (TR^{T,P}), the partial transformed target representation (TR^{T,P}) being reduced to one part or multiple parts of the transformed target representation (TR^{T});
wherein the generating and/or receiving of a transformed synthetic representation (SR^{T}) on the basis of and/or in relation to the synthetic representation (SR^{T}) comprises:
generating a partial transformed synthetic representation (SR^{T,P}), the partial transformed synthetic representation (SR^{T,P}) being reduced to one part or multiple parts of the transformed synthetic representation (SR^{T}); and
wherein the quantifying of the differences between the transformed synthetic representation (SR^{T}) and the transformed target representation (TR^{T}) comprises:
quantifying the differences between the partial transformed synthetic representation (SR^{T,P}) and the partial transformed target representation (TR^{T,P}).

4. The method as claimed in claim 2,
wherein the training comprises for each examination object of the multiplicity of examination objects:
feeding the at least one input representation (R1, R2) to the machine-learning model;
receiving the synthetic representation (SR) and a first transformed synthetic representation (SR^{T}) of the examination region of the examination object from the machine-learning model (MLM), the first transformed synthetic representation (SR^{T}) representing at least part of the examination region of the examination object;
in frequency space, if the synthetic representation (SR) represents the examination region of the examination object in real space; or
in real space, if the synthetic representation (SR) represents the examination region of the examination object in frequency space;
generating a second transformed synthetic representation (SR^{T #}) on the basis of the synthetic representation (SR) by means of a transform (T), the second transformed synthetic representation (SR^{T #}) representing at least part of the examination region of the examination object;
in frequency space, if the synthetic representation (SR) represents the examination region of the examination object in real space; or
in real space, if the synthetic representation (SR) represents the examination region of the examination object in frequency space;
quantifying the differences i) between at least part of the synthetic representation (SR) and at least part of the target representation (SR), ii) between at least part of the first transformed synthetic representation (SR^{T}) and at least part of the transformed target representation (TR^{T}) and iii) between at least part of the first transformed synthetic representation (SR^{T}) and at least part of the second transformed synthetic representation (SR^{T #}) by means of a loss function (L); and
reducing the differences by modifying model parameters (MP).

5. The method as claimed in claim 1,
wherein the machine-learning model (MLM) undergoing training comprises a first machine-learning model (MLM1) and a second machine-learning model (MLM2);
wherein the first machine-learning model (MLM1) is configured to generate on the basis of at least one input representation (R1, R2) and model parameters (MP1) a synthetic representation (SR) of the examination region of the examination object;
wherein the second machine-learning model (MLM2) is configured to reconstruct on the basis of the synthetic representation (SR) of the examination region of the examination object and model parameters (MP2) at least one input representation (R1, R2);
wherein the training comprises for each examination object of the multiplicity of examination objects:
generating a transformed input representation (R2) on the basis of at least one input representation (R1) by means of a transform (T), the transformed input representation (R2) representing at least part of the examination region of the examination object;
in frequency space, if the at least one input representation (R1) represents the examination region of the examination object in real space; or
in real space, if the at least one input representation (R1) represents the examination region of the examination object in frequency space;
feeding the at least one input representation (R1) and/or the transformed input representation (R2) to the first machine-learning model;
receiving a synthetic representation (SR) of the examination region of the examination object from the first machine-learning model (MLM1); and
generating and/or receiving a transformed synthetic representation (SR) on the basis of and/or in relation to the synthetic representation (SR), the transformed synthetic representation (SR) representing at least part of the examination region of the examination object;
in frequency space, if the synthetic representation (SR) represents the examination region of the examination object in real space; or
in real space, if the synthetic representation (SR) represents the examination region of the examination object in frequency space;
feeding the synthetic representation (SR) and/or the transformed synthetic representation (SR^{T}) to the second machine-learning model (MLM2);
receiving a predicted input representation (R1 #) from the second machine-learning model (MLM2); and
generating and/or receiving a transformed predicted input representation (R2 #) on the basis of and/or in relation to the predicted input representation (R1 #), the transformed predicted input representation (R2 #) representing at least part of the examination region of the examination object;
in frequency space, if the predicted input representation (R1 #) represents the examination region of the examination object in real space; or
in real space, if the predicted input representation (R1 #) represents the examination region of the examination object in frequency space;
quantifying the differences i) between at least part of the synthetic representation (SR) and at least part of the target representation (TR), ii) between at least part of the transformed synthetic representation (SR^{T}) and at least part of the transformed target representation (TR^{T}), iii) between at least part of the input representation (R1) and at least part of the predicted input representation (R1 #) and iv) between at least part of the transformed input representation (R2) and at least part of the transformed predicted input representation (R2 #) by means of a loss function (L); and
reducing the differences by modifying model parameters (MP).

6. The method as claimed in claim 1, wherein the first amount of the contrast agent is lower than the second amount of the contrast agent.

7. The method as claimed in claim 1, wherein the examination object is a human.

8. The method as claimed in claim 1, wherein the examination region includes a liver, brain, heart, kidney, lung, stomach, intestine, pancreas, thyroid gland, prostate or breast of a human.

9. The method as claimed in claim 1, wherein each input representation of the at least one input representation (R1, R2, R1*, R2*) is a representation of the examination region in real space, the target representation (TR) is a representation of the examination region in real space, and the synthetic representation (SR, SR*) is a representation of the examination region in real space.

10. The method as claimed in claim 3, wherein the partial transformed synthetic representation (SR^{T,P}) represents the examination region in frequency space, the partial transformed synthetic representation (SR^{T,P}) being reduced to a frequency range of the transformed synthetic representation (SR^{T}), contrast information being encoded in the frequency range.

11. The method as claimed in claim 3, wherein the partial transformed synthetic representation (SR^{T,P}) represents the examination region in frequency space, the partial transformed synthetic representation (SR^{T,P}) being reduced to a frequency range of the transformed synthetic representation (SR^{T}), information about fine structures being encoded in the frequency range.

12. A computer system comprising:
a receiving unit;
a control and calculation unit; and
an output unit;
wherein the control and calculation unit is configured to provide a trained machine-learning model (MLM^{t});
the trained machine-learning model (MLM^{t}) having been trained by means of training data (TD) to generate on the basis of at least one input representation (R1, R2) of an examination region of an examination object a synthetic representation (SR) of the examination region of the examination object;
the training data (TD) comprising for each examination object of a multiplicity of examination objects i) an input representation (R1, R2) of the examination region of the examination object, ii) a target representation (TR) of the examination region and iii) a transformed target representation (TR^{T});
the at least one input representation (R1, R2) representing the examination region of the respective examination object without contrast agent or after administration of a first amount of a contrast agent;
the target representation (TR) representing the examination region of the respective examination object after administration of a second amount of the contrast agent; and
the transformed target representation (TR^{T}) representing at least part of the examination region of the respective examination object in frequency space, if the target representation (TR) represents the examination region of the respective examination object in real space, or in real space, if the target representation (TR) represents the examination region of the respective examination object in frequency space;
the training of the machine-learning model (MLM^{t}) comprising reducing differences between i) at least part of the synthetic representation (SR) and at least part of the target representation (TR) and ii) at least part of a transformed synthetic representation (SR^{T}) and at least part of the transformed target representation (TR);
wherein the control and calculation unit is configured to cause the receiving unit to receive at least one input representation (R1*, R2*) of an examination region of a new examination object, the at least one input representation (R1*, R2*) representing the examination region without and/or after administration of the first amount of the contrast agent;
wherein the control and calculation unit is configured to input the received at least one input representation (R1*, R2*) into the trained machine-learning model (MLM);
wherein the control and calculation unit is configured to receive from the trained machine-learning model (MLM^{t}) a synthetic representation (SR*) of the examination region of the new examination object; and
wherein the control and calculation unit is configured to cause the output unit to output the synthetic representation (SR*) of the examination region of the new examination object and/or to store it and/or to transmit it to a separate computer system.

13. A computer program product comprising a computer program that can be loaded into a working memory of a computer system, wherein it causes the computer system to execute the following:
providing a trained machine-learning model (MLM^{t});
the trained machine-learning model (MLM^{t}) having been trained by means of training data (TD) to generate on the basis of at least one input representation (R1, R2) of an examination region of an examination object a synthetic representation (SR) of the examination region of the examination object;
the training data (TD) comprising for each examination object of a multiplicity of examination objects i) an input representation (R1, R2) of the examination region of the examination object, ii) a target representation (TR) of the examination region of the examination object and iii) a transformed target representation (TR^{T});
the at least one input representation (R1, R2) representing the examination region of the respective examination object without or after administration of a first amount of a contrast agent;
the target representation (TR) representing the examination region of the respective examination object after administration of a second amount of the contrast agent;
the transformed target representation (TR^{T}) representing at least part of the examination region of the examination object in frequency space, if the target representation (TR) represents the examination region of the examination object in real space, or in real space, if the target representation (TR) represents the examination region of the examination object in frequency space;
the training of the machine-learning model (MLM^{t}) comprising reducing the differences between i) at least part of the synthetic representation (SR) and at least part of the target representation (TR) and ii) between at least part of a transformed synthetic representation (SR^{T}) and at least part of the transformed target representation (TR^{T}); and
receiving at least one input representation (R1*, R2*) of an examination region of a new examination object, the at least one input representation (R1*, R2*) representing the examination region of the new examination object without the contrast agent and/or after administration of a first amount of the contrast agent;
inputting the at least one input representation (R1*, R2*) of the examination region of the new examination object into the trained machine-learning model (MLM^{t});
receiving a synthetic representation (SR*) of the examination region of the new examination object from the trained machine-learning model (MLM^{t}); and
outputting and/or storing the synthetic representation (SR*) of the examination region of the new examination object and/or transmitting the synthetic representation (SR*) of the examination region of the new examination object to a separate computer system.

14.-16. (canceled)

17. The method as claimed in claim 1, wherein the contrast agent comprises one or more contrast agents selected from the following:
gadoxetate disodium;
gadolinium(III) 2-[4,7,10-tris(carboxymethyl)-1,4,7,10-tetrazacyclododec-1-yl]acetic acid;
gadolinium(III) ethoxybenzyldiethylenetriaminepentaacetic acid;
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;
dihydrogen [(±)-4-carboxy-5,8,11-tris(carboxymethyl)-1-phenyl-2-oxa-5,8,11-triazatridecan-13-oato(5-)]gadolinate(2-);
tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate, gadolinium 2,2′,2″-(10-{1-carboxy-2-[2-(4-ethoxyphenyl)ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate;
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate);
gadolinium 2,2′,2″-{10-[(1S)-4-(4-butoxyphenyl)-1-carboxybutyl]-1,4,7,10-tetraazacyclododecane-1,4,7-triyl}triacetate;
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate;
gadolinium-2,2′,2″-[10-(carboxymethyl)-2-(4-ethoxybenzyl)-1,4,7,10-tetraazacyclododecane-1,4,7-triyl]triacetate;
gadolinium(III) 5,8-bis(carboxylatomethyl)-2-[2-(methylamino)-2-oxoethyl]-10-oxo-2,5,8,11-tetraazadodecane-1-carboxylate hydrate;
gadolinium(III) 2-[4-(2-hydroxypropyl)-7,10-bis(2-oxido-2-oxoethyl)-1,4,7,10-tetrazacyclododec-1-yl]acetate;
gadolinium(III) 2,2′,2″-(10-((2R,3S)-1,3,4-trihydroxybutan-2-yl)-1,4,7,10-tetraazacyclododecane-1,4,7-triyl)triacetate;
a Gd³⁺ complex of a compound of the formula (I) 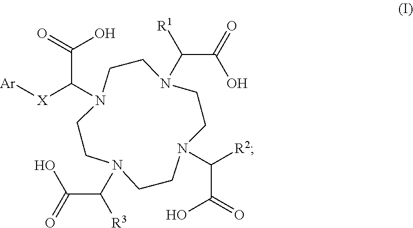
wherein
Ar is a group selected from 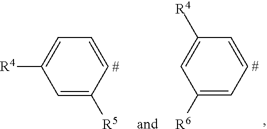
wherein #is the linkage to X;
X is a group selected from
CH₂, (CH₂)₂, (CH₂)₃, (CH₂)₄ and *—(CH₂)₂O—CH₂-^{#};
wherein * is the linkage to Ar and #is the linkage to the acetic acid residue;
R¹, R² and R³ are each independently a hydrogen atom or a group selected from C₁-C₃ alkyl, —CH₂OH, —(CH₂)₂OH and —CH₂OCH₃;
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—;
R⁵ is a hydrogen atom;
and
R⁶ is a hydrogen atom;
or a stereoisomer, tautomer, hydrate, solvate or salt thereof, or a mixture thereof;
a Gd³⁺ complex of a compound of the formula (II) 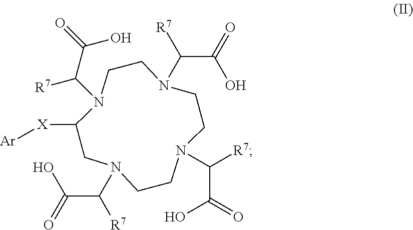
wherein
Ar is a group selected from 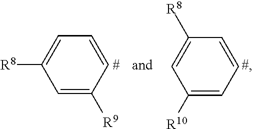
wherein #is the linkage to X;
X is a group selected from CH₂, (CH₂)₂, (CH₂)₃, (CH₂)₄ and *—(CH₂)₂O—CH₂— #, wherein * is the linkage to Ar and #is the linkage to the acetic acid residue;
R⁷ is a hydrogen atom or a group selected from C₁-C₃ alkyl, —CH₂OH, —(CH₂)₂OH and —CH₂OCH₃;
R⁸ is a group selected from C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

18. The computer system as claimed in claim 12, wherein the contrast agent comprises one or more contrast agents selected from the following:
gadoxetate disodium;
gadolinium(III) 2-[4,7,10-tris(carboxymethyl)-1,4,7,10-tetrazacyclododec-1-yl]acetic acid, gadolinium(III) ethoxybenzyldiethylenetriaminepentaacetic acid;
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;
dihydrogen [(±)-4-carboxy-5,8,11-tris(carboxymethyl)-1-phenyl-2-oxa-5,8,11-triazatridecan-13-oato(5-)]gadolinate(2-);
tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate, gadolinium 2,2′,2″-(10-{1-carboxy-2-[2-(4-ethoxyphenyl)ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate;
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate);
gadolinium 2,2′,2″-{10-[(1S)-4-(4-butoxyphenyl)-1-carboxybutyl]-1,4,7,10-tetraazacyclododecane-1,4,7-triyl}triacetate;
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate;
gadolinium-2,2′,2″-[10-(carboxymethyl)-2-(4-ethoxybenzyl)-1,4,7,10-tetraazacyclododecane-1,4,7-triyl]triacetate;
gadolinium(III) 5,8-bis(carboxylatomethyl)-2-[2-(methylamino)-2-oxoethyl]-10-oxo-2,5,8,11-tetraazadodecane-1-carboxylate hydrate;
gadolinium(III) 2-[4-(2-hydroxypropyl)-7,10-bis(2-oxido-2-oxoethyl)-1,4,7,10-tetrazacyclododec-1-yl]acetate;
gadolinium(III) 2,2′,2″-(10-((2R,3S)-1,3,4-trihydroxybutan-2-yl)-1,4,7,10-tetraazacyclododecane-1,4,7-triyl)triacetate;
a Gd³⁺ complex of a compound of the formula (I) 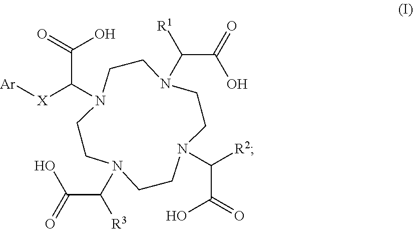
wherein
Ar is a group selected from 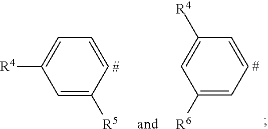
wherein #is the linkage to X;
X is a group selected from
CH₂, (CH₂)₂, (CH₂)₃, (CH₂)₄ and *—(CH₂)₂O—CH₂-^{#};
wherein * is the linkage to Ar and #is the linkage to the acetic acid residue;
R¹, R² and R³ are each independently a hydrogen atom or a group selected from C₁-C₃ alkyl, —CH₂OH, —(CH₂)₂OH and —CH₂OCH₃;
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—;
R⁵ is a hydrogen atom;
and
R⁶ is a hydrogen atom;
or a stereoisomer, tautomer, hydrate, solvate or salt thereof, or a mixture thereof,
a Gd³⁺ complex of a compound of the formula (II) 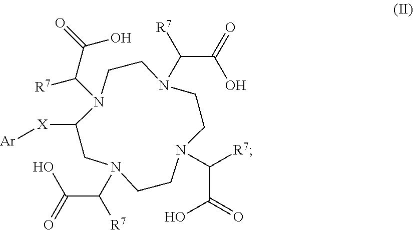
wherein
Ar is a group selected from 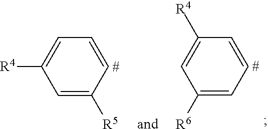
wherein #is the linkage to X;
X is a group selected from CH₂, (CH₂)₂, (CH₂)₃, (CH₂)₄ and *—(CH₂)₂O—CH₂-^{#}, wherein * is the linkage to Ar and #is the linkage to the acetic acid residue;
R⁷ is a hydrogen atom or a group selected from C₁-C₃ alkyl, —CH₂OH, —(CH₂)₂OH and —CH₂OCH₃;
R⁸ is a group selected from C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

19. The computer program product as claimed in claim 13, wherein the contrast agent comprises one or more contrast agents selected from the following:
gadoxetate disodium;
gadolinium(III) 2-[4,7,10-tris(carboxymethyl)-1,4,7,10-tetrazacyclododec-1-yl]acetic acid;
gadolinium(III) ethoxybenzyldiethylenetriaminepentaacetic acid;
gadolinium(III) 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;
dihydrogen [(±)-4-carboxy-5,8,11-tris(carboxymethyl)-1-phenyl-2-oxa-5,8,11-triazatridecan-13-oato(5-)]gadolinate(2-);
tetragadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate, gadolinium 2,2′,2″-(10-{1-carboxy-2-[2-(4-ethoxyphenyl)ethoxy]ethyl}-1,4,7,10-tetraazacyclododecane-1,4,7-triyl)triacetate;
gadolinium 2,2′,2″-{10-[1-carboxy-2-{4-[2-(2-ethoxyethoxy)ethoxy]phenyl}ethyl]-1,4,7,10-tetraazacyclododecane-1,4,7-triyl}triacetate;
gadolinium 2,2′,2″-{10-[(1R)-1-carboxy-2-{4-[2-(2-ethoxyethoxy)ethoxy]phenyl}ethyl]-1,4,7,10-tetraazacyclododecane-1,4,7-triyl}triacetate;
gadolinium (2S,2′S,2″S)-2,2′,2″-{10-[(1S)-1-carboxy-4-{4-[2-(2-ethoxyethoxy)ethoxy]phenyl}butyl]-1,4,7,10-tetraazacyclododecane-1,4,7-triyl}tris(3-hydroxypropanoate);
gadolinium 2,2′,2″-{10-[(1S)-4-(4-butoxyphenyl)-1-carboxybutyl]-1,4,7,10-tetraazacyclododecane-1,4,7-triyl}triacetate;
gadolinium-2,2′,2″-{(2S)-10-(carboxymethyl)-2-[4-(2-ethoxyethoxy)benzyl]-1,4,7,10-tetraazacyclododecane-1,4,7-triyl}triacetate;
gadolinium-2,2′,2″-[10-(carboxymethyl)-2-(4-ethoxybenzyl)-1,4,7,10-tetraazacyclododecane-1,4,7-triyl]triacetate;
gadolinium(III) 5,8-bis(carboxylatomethyl)-2-[2-(methylamino)-2-oxoethyl]-10-oxo-2,5,8,11-tetraazadodecane-1-carboxylate hydrate;
gadolinium(III) 2-[4-(2-hydroxypropyl)-7,10-bis(2-oxido-2-oxoethyl)-1,4,7,10-tetrazacyclododec-1-yl]acetate;
gadolinium(III) 2,2′,2″-(10-((2R,3S)-1,3,4-trihydroxybutan-2-yl)-1,4,7,10-tetraazacyclododecane-1,4,7-triyl)triacetate;
a Gd³⁺ complex of a compound of the formula (I) 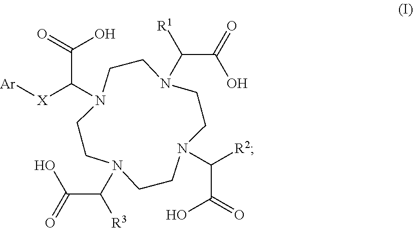
wherein
Ar is a group selected from 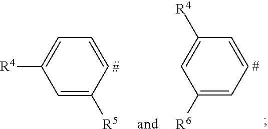
wherein #is the linkage to X;
X is a group selected from CH₂, (CH₂)₂, (CH₂)₃, (CH₂)₄ and *—(CH₂)₂O—CH₂—;
wherein * is the linkage to Ar and #is the linkage to the acetic acid residue;
R¹, R² and R³ are each independently a hydrogen atom or a group selected from C₁-C₃ alkyl, —CH₂OH, —(CH₂)₂OH and —CH₂OCH₃;
R⁴ is a group selected from C₂-C₄ alkoxy, (H₃C—CH₂)—O—(CH₂)₂—O—, (H₃C—CH₂)—O—(CH₂)₂—O—(CH₂)₂—O— and (H₃C—CH₂)—O—(CH₂)₂—O—(CH₂)₂—O—(CH₂)₂—O—;
R⁵ is a hydrogen atom;
and
R⁶ is a hydrogen atom;
or a stereoisomer, tautomer, hydrate, solvate or salt thereof, or a mixture thereof,
a Gd³⁺ complex of a compound of the formula (II) 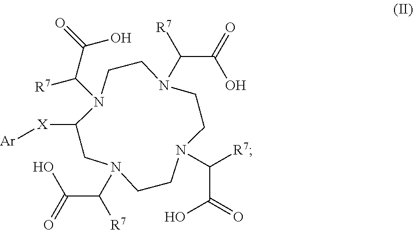
wherein
Ar is a group selected from 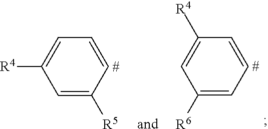
wherein #is the linkage to X;
X is a group selected from CH₂, (CH₂)₂, (CH₂)₃, (CH₂)₄ and *—(CH₂)₂—O—CH₂-^{#}, wherein * is the linkage to Ar and #is the linkage to the acetic acid residue;
R⁷ is a hydrogen atom or a group selected from C₁-C₃ alkyl, —CH₂OH, —(CH₂)₂OH and —CH₂OCH₃;
R⁸ is a group selected from C₂-C₄ alkoxy, (H₃C—CH₂O)—(CH₂)₂—O—, (H₃C—CH₂O)—(CH₂)₂—O—(CH₂)₂—O— and (H₃C—CH₂O)—(CH₂)₂—O—(CH₂)₂—O—(CH₂)₂—O—;
R⁹ and R¹⁰ are each independently a hydrogen atom;
or a stereoisomer, tautomer, hydrate, solvate or salt thereof, or a mixture thereof.

20. The computer program product as claimed in claim 13,
wherein the training of the machine-learning model (MLM^{t}) comprises:
receiving and/or providing the training data (TD), the training data (TD) comprising a set of input data and target data for each examination object of the multiplicity of examination objects;
each set comprising the at least one input representation (R¹, R²) of the examination region of the examination object as input data and the target representation (TR) of the examination region of the examination object and the transformed target representation (TR^{T}) as target data;
the at least one input representation (R¹, R²) representing the examination region without contrast agent and/or after administration of the first amount of the contrast agent and the target representation (TR) representing the examination region after the administration of the second amount of the contrast agent; and
the transformed target representation (TR^{T}) representing at least part of the examination region of the examination object;
in frequency space, if the target representation (TR) represents the examination region of the examination object in real space; or
in real space, if the target representation (TR) represents the examination region of the examination object in frequency space;
receiving and/or providing a machine-learning model (MLM), the machine-learning model (MLM) being configured to generate on the basis of at least one input representation (R1, R2) of an examination region of an examination object and model parameters (MP) a synthetic representation (SR) of the examination region of the examination object; and
training the machine-learning model (MLM), the training comprising for each examination object of the multiplicity of examination objects;
feeding the at least one input representation (R1, R2) of the examination region of the examination object to the machine-learning model (MLM);
receiving a synthetic representation (SR) of the examination region of the examination object from the machine-learning model (MLM); and
generating and/or receiving a transformed synthetic representation (SR^{T}) on the basis of the synthetic representation (SR) and/or in relation to the synthetic representation (SR), the transformed synthetic representation (SR^{T}) representing at least part of the examination region of the examination object;
in frequency space, if the synthetic representation (SR) represents the examination region of the examination object in real space; or
in real space, if the synthetic representation (SR) represents the examination region of the examination object in frequency space;
quantifying the differences i) between at least part of the synthetic representation (SR) and at least part of the target representation (TR) and ii) between at least part of the transformed synthetic representation (SR^{T}) and at least part of the transformed target representation (TR^{T}) by means of a loss function (L); and
reducing the differences by modifying model parameters (MP);
outputting and/or storing the trained machine-learning model (MLM) and/or transmitting the trained machine-learning model (MLM) to a separate computer system.

21. The computer program product as claimed in claim 13,
wherein the training comprises for each examination object of the multiplicity of examination objects:
feeding the at least one input representation (R1, R2) to the machine-learning model;
receiving the synthetic representation (SR) and a first transformed synthetic representation (SR^{T}) of the examination region of the examination object from the machine-learning model (MLM), the first transformed synthetic representation (SR) representing at least part of the examination region of the examination object;
in frequency space, if the synthetic representation (SR) represents the examination region of the examination object in real space; or
in real space, if the synthetic representation (SR) represents the examination region of the examination object in frequency space;
generating a second transformed synthetic representation (SR^{T}*) on the basis of the synthetic representation (SR) by means of a transform (7), the second transformed synthetic representation (SR^{T}*) representing at least part of the examination region of the examination object;
in frequency space, if the synthetic representation (SR) represents the examination region of the examination object in real space; or
in real space, if the synthetic representation (SR) represents the examination region of the examination object in frequency space;
quantifying the differences i) between at least part of the synthetic representation (SR) and at least part of the target representation (SR), ii) between at least part of the first transformed synthetic representation (SR) and at least part of the transformed target representation (TR^{T}) and iii) between at least part of the first transformed synthetic representation (SR^{T}) and at least part of the second transformed synthetic representation (SR^{T #}) by means of a loss function (L); and
reducing the differences by modifying model parameters (MP).

22. The computer program product as claimed in claim 13, wherein the machine-learning model (MLM) undergoing training comprises a first machine-learning model (MLM1) and a second machine-learning model (MLM2);
wherein the first machine-learning model (MLM1) is configured to generate on the basis of at least one input representation (R1, R2) and model parameters (MP1) a synthetic representation (SR) of the examination region of the examination object;
wherein the second machine-learning model (MLM2) is configured to reconstruct on the basis of the synthetic representation (SR) of the examination region of the examination object and model parameters (MP2) at least one input representation (R1, R2);
wherein the training comprises for each examination object of the multiplicity of examination objects:
generating a transformed input representation (R2) on the basis of at least one input representation (R1) by means of a transform (7), the transformed input representation (R2) representing at least part of the examination region of the examination object;
in frequency space, if the at least one input representation (R1) represents the examination region of the examination object in real space; or
in real space, if the at least one input representation (R1) represents the examination region of the examination object in frequency space;
feeding the at least one input representation (R1) and/or the transformed input representation (R2) to the first machine-learning model;
receiving a synthetic representation (SR) of the examination region of the examination object from the first machine-learning model (MLM1); and
generating and/or receiving a transformed synthetic representation (SR) on the basis of and/or in relation to the synthetic representation (SR), the transformed synthetic representation (SR^{T}) representing at least part of the examination region of the examination object;
in frequency space, if the synthetic representation (SR) represents the examination region of the examination object in real space; or
in real space, if the synthetic representation (SR) represents the examination region of the examination object in frequency space;
feeding the synthetic representation (SR) and/or the transformed synthetic representation (SR) to the second machine-learning model (MLM2);
receiving a predicted input representation (R1 #) from the second machine-learning model (MLM2); and
generating and/or receiving a transformed predicted input representation (R2 #) on the basis of and/or in relation to the predicted input representation (R1 #), the transformed predicted input representation (R2 #) representing at least part of the examination region of the examination object;
in frequency space, if the predicted input representation (R1 #) represents the examination region of the examination object in real space; or
in real space, if the predicted input representation (R1 #) represents the examination region of the examination object in frequency space;
quantifying the differences i) between at least part of the synthetic representation (SR) and at least part of the target representation (TR), ii) between at least part of the transformed synthetic representation (SR^{T}) and at least part of the transformed target representation (TR^{T}), iii) between at least part of the input representation (R1) and at least part of the predicted input representation (R1 #) and iv) between at least part of the transformed input representation (R2) and at least part of the transformed predicted input representation (R2 #) by means of a loss function (L); and
reducing the differences by modifying model parameters (MP).

23. The computer program product as claimed in claim 13, wherein the first amount of the contrast agent is different from the second amount of the contrast agent.
